SEQUENCE LISTING (8 sequences)

SEQ ID NO: 1 - protein, 12 aa, Artificial Sequence
  circular or linear NLS
CVQRKRQKLMPC

SEQ ID NO: 2 - protein, 12 aa, Artificial Sequence
  aMTD385
IVAIAVPALVAP

SEQ ID NO: 3 - protein, 12 aa, Artificial Sequence
  aMTD666
AAIAIIAPAIVP

SEQ ID NO: 4 - protein, 12 aa, Artificial Sequence
  aMTD827
IAAVLAAPALVP

SEQ ID NO: 5 - protein, 12 aa, Artificial Sequence
  aMTD831
IIVAVAPAAIVP

SEQ ID NO: 6 - protein, 12 aa, Artificial Sequence
  aMTD891
ILAVAAIPAALP

SEQ ID NO: 7 - protein, 16 aa, Artificial Sequence
  CPP of cSN50.1
AAVALLPAVLLALLAP

SEQ ID NO: 8 - protein, 10 aa, Artificial Sequence
  NLS
VQRKRQKLMP

(19) United States
(12) Patent Application Publication
(10) Pub. No.: US 2023/0039219 A1
(43) Pub. Date: Feb. 9, 2023

(54) Improved Cell-Permeable Nuclear Import Inhibitor Synthetic Peptide for Inhibition of Cytokine Storm or Inflammatory Disease and Use Thereof

(71) Applicant: Cellivery Therapeutics, Inc., Seoul (KR)

(72) Inventor: Daewoong Jo, Seoul (KR)

(21) Appl. No.: 17/783,822

(22) PCT Filed: Feb. 16, 2021
(86) PCT No.: PCT/KR2021/001976

§ 371 Date: Jun. 9, 2022

Related U.S. Application Data
(60) Provisional application No. 62/977,832, filed on Feb. 18, 2020.

(51) Int. Cl. C07K 14/47; A61P 29/00

(52) U.S. Cl. CPC C07K 14/4703; A61P 29/00; C07K 2319/09

(57) ABSTRACT

Provided is an improved cell-permeable nuclear import inhibitor (iCP-NI) for inhibition of cytokine storm or an inflammatory disease, in which solubility and stability are improved by introducing an advanced macromolecule transduction domain (aMTD)-based therapeuticmolecule systemic delivery technology (TSDT) into a cell-permeable nuclear import inhibitor (CP-NI, cSN50.1 peptide). The improved cell-permeable nuclear import inhibitor synthetic peptide according to the present disclosure more efficiently blocks signal transduction mediated by stress-responsive transcription factors (SRTFs) including NF-κB, based on remarkable cell permeability, and thus it may be used as an excellent prophylactic or therapeutic agent for cytokine storm or inflammatory diseases.

### TECHNICAL FIELD

The present disclosure relates to an improved cell-permeable nuclear import inhibitor (iCP-NI) synthetic peptide for inhibition of cytokine storm or an inflammatory disease, in which solubility and stability are improved by introducing an advanced macromolecule transduction domain (aMTD)-based therapeuticmolecule systemic delivery technology (TSDT) into a cell-permeable nuclear import inhibitor (CP-NI, cSN50.1 peptide). The improved cell-permeable nuclear import inhibitor synthetic peptide according to the present disclosure more efficiently blocks signal transduction mediated by stress-responsive transcription factors (SRTFs) including NF-κB, based on remarkable cell permeability, and thus it may be used as an excellent prophylactic or therapeutic agent for cytokine storm or inflammatory diseases.

### BACKGROUND ART

Inflammatory responses refer to a defense mechanism to protect a living body from microbial infection or external damage. The purpose of inflammation is to suppress cell injury in the early stages, to remove damaged tissues and necrotic cells from the wound, and at the same time, to initiate tissue regeneration. Inflammation itself is not a disease, but rather corresponds to a self-defense system necessary for living organisms. However, when the body's defense system is excessively activated, a phenomenon called “cytokine storm” occurs due to an uncontrolled immune response. Cytokine storm exhibits a severe inflammatory response throughout the body, resulting in vasodilation, fever, release of acute phase proteins by the liver, and recruitment of leukocytes towards inflammatory foci, and in severe cases, eventually leading to death of the patient due to hypotension and organ failure. In addition, excessive inflammation and cytokine storm are known as the cause of several inflammatory diseases.

Sepsis, which is one of inflammatory disorders, is a symptom caused by bacterial or viral infection, severe trauma, etc., and causative substances, such as a pathogen-associated molecule pattern (PAMP) derived from a pathogen that has invaded the body, or a damage-associated molecule pattern (DAMP) derived from a damaged tissue, cause excessive activation of the self-defense system such as macrophages and cytokine storm. This excessive systemic inflammatory response causes abnormalities in the circulatory system, and decreases blood pressure by increased vascular permeability due to capillary leaks, eventually leading to multiple organ failure (MOF) due to insufficient supply of blood to various organs throughout the body. Sepsis is a representative intractable disease with a mortality rate of about 30%, and 27 million patients occur every year worldwide. Sepsis is ranked as the third cause of death in the world, after cancer and heart disease. Therefore, blockade of cytokine storm by inhibiting inflammatory responses in the body and cells should be a target of sepsis treatment.

In addition, Severe acute respiratory syndrome coronavirus 2 (SARS-CoV-2), the causative agent of coronavirus disease 2019 was first reported in December 2019. Since the initial cases of COVID-19 were reported from Wuhan, China in December 2019 (Huang, C. et al. Clinical features of patients infected with 2019 novel coronavirus in Wuhan, China. Lancet 395, 497-506 (2020)), SARS-CoV-2 has emerged as a global pandemic with an ever-increasing number of severe cases requiring invasive external ventilation that threatens to overwhelm health care systems (World Health Organization. Coronavirus disease (COVID-2019) situation reports. See website made up of “https://www.” before “who.int/emergencies/disease/novel-coronavirus-2019/situation-reports”). While it remains unclear why COVID-19 patients experience a spectrum of clinical outcomes ranging from asymptomatic to severe disease, the salient features of COVID-19 pathogenesis and mortality are rampant inflammation and CRS leading to ARDS (Mehta, P. et al. COVID-19: consider cytokine storm syndromes and immunosuppression. Lancet 395, 1033-1034 (2020); Qin, C. et al. Dysregulation of immune response in patients with COVID-19 in Wuhan, China. Clin. Infect. Dis. (2020)). Indeed, excessive immune cell infiltration into the lung, cytokine storm, and ARDS have previously been described as defining features of severe disease in humans infected with the closely related betacoronaviruses SARS-CoV.

Meanwhile, the amplification of proinflammatory signals relies on a limited number of transcription factors (here designated as stress-responsive transcription factors, SRTFs), including nuclear factor kappa B (NF-κB), activator protein 1 (AP-1), signal transducer and activator of transcription (STAT) and nuclear factor of activated T cells (NFAT) that regulate the expression of cytokines and other inflammatory genes in response to signals initiated by PRRs and proinflammatory cytokine receptors. To respond rapidly to inflammation, cells of the innate immune system maintain pools of SRTFs sequestered in the cytoplasm in an inactive state. The SRTFs are activated by phosphorylation-dependent changes that unmask a nuclear localization sequence (NLS) thereby allowing the proteins to be shuttled into the nucleus. NF-κB, is so activated by phosphorylation of an inhibitory protein (IkB); AP-1 and STAT1/3 are activated by phosphorylation; and NFAT1 by dephosphorylation.

Previous studies tested the possibility of inhibiting inflammatory responses by targeting SRTF nuclear transport. The task was simplified by the fact that the NLS of each transcription factor was recognized by the same transport adaptor protein, Importin-5α. A cell-permeable peptide (cSN50.1) was synthesized that delivered sufficient number of NLS sequences into cells to competitively inhibit nuclear import of all 4 SRTFs and block lipopolysaccharide (LPS)-dependent activation of cytokine gene expression. Moreover, cSN50.1 was able to protect mice challenged with lethal doses of LPS and other proinflammatory agonists.

### DISCLOSURE OF INVENTION

### Technical Problem

The present inventors have made extensive efforts to overcome the limitations of the existing cell-permeable nuclear import inhibitor (CP-NI, cSN50.1 peptide), and as a result, they found that when an therapeuticmolecule systemic delivery technology (TSDT)-based advanced macromolecule transduction domain (aMTD) is introduced into CP-NI solubility and stability may be improved, thereby completing the present disclosure.

### Solution to Problem

An object of the present disclosure is to provide an improved cell-permeable nuclear import inhibitor (iCP-NI) synthetic peptide for inhibition of cytokine storm or an inflammatory disease, the iCP-NI synthetic peptide including an NF-κB nuclear localization sequence (NLS) and an advanced macromolecule transduction domain (aMTD), wherein the NF-κB nuclear localization sequence includes an amino acid sequence of SEQ ID NO: 1, and the advanced macromolecule transduction domain includes an amino acid sequence selected from the group consisting of SEQ ID NOs: 2 to 6. The iCP-NI synthetic peptide according to the present disclosure binds with impotin α to inhibit nuclear translocation of stress-responsive transcription factors (SRTFs) including NF-κB, thereby preventing generation of cytokine storm in advance.

Another object of the present disclosure is to provide a pharmaceutical composition for preventing or treating cytokine storm or an inflammatory disease, the pharmaceutical composition including the iCP-NI synthetic peptide.

Still another object of the present disclosure is to provide a method of preventing or treating cytokine storm or an inflammatory disease, the method including the step of administering, to a subject, the iCP-NI synthetic peptide including the NF-κB nuclear localization sequence and the advanced macromolecule transduction domain.

### Advantageous Effects of Invention

An improved cell-permeable nuclear import inhibitor (iCP-NI) synthetic peptide according to the present disclosure more efficiently blocks signal transduction mediated by stress-responsive transcription factors (SRTFs) including NF-κB, based on remarkable cell permeability, and thus it is expected that the iCP-NI synthetic peptide may be used as an excellent prophylactic or therapeutic agent for cytokine storm or inflammatory diseases.

### BRIEF DESCRIPTION OF DRAWINGS

FIG. 1 shows an effect of aMTDs-NLSs peptides on a survival rate in LPS/D-Gal acute liver injury model;

FIG. 2 shows an effect of the NLS sequence alone in a linear (1NLS) or circular (cNLS) form on a survival rate of in LPS/D-Gal acute liver injury model;

FIG. 3 shows an effect of intravenous (IV) or intraperitoneal (IP) administration of aMTD827-cNLS on a survival rate in LPS/D-Gal acute liver injury model;

FIG. 4 shows an effect of the cSN50.1 peptide on a survival rate in LPS/D-Gal acute liver injury model;

FIG. 5 shows a therapeutic effect on a survival rate of iCP-NI in LPS/D-Gal acute liver injury model;

FIG. 6 shows a stability of iCP-NI for over 3 months at 25° C.

FIG. 7 shows an ability of iCP-NI to bind Importin-a5 as assessed by surface plasmon resonance;

FIG. 8 shows a result of flow cytometry for iCP-NI conjugated with fluorescein isothiocyanate (FITC) in cultured RAW264.7 cells;

FIG. 9 shows a distribution of FITC-iCP-NI in lungs brain, heart, liver, spleen, and kidney;

FIG. 10 shows a pharmacokinetic of FITC-iCP-NI in lung tissue;

FIG. 11 shows an effect of iCP-NI on nuclear translocation of NF-κB, AP-1 and STAT3 by Western Blot analysis of nuclear and cytoplasmic fractions;

FIG. 12 shows nuclear translocation inhibitory effect of iCP-NI of NF-κB, AP-1 and STAT3 by immunostaining;

FIG. 13 shows nuclear translocation inhibitory effect iCP-NI of NF-κB, STAT1/3, AP-1 and NFAT by Western Blot analysis of nuclear and cytoplasmic fractions;

FIG. 14 shows a nuclear translocation inhibitory effect of iCP-NI of NF-κB, STAT1/3, AP-1 and NFAT by immunostaining;

FIG. 15 shows an effect of iCP-NI on a survival rate in LPS/D-Gal acute liver injury model;

FIG. 16 shows an effect of iCP-NI on the expression of TNF-α, IL-6 and IL-10;

FIG. 17 shows an effect of iCP-NI on liver damage and massive liver apoptosis;

FIG. 18 shows an effect of iCP-NI on a survival rate in LPS/Poly I:C induced pneumonitis model;

FIG. 19 shows an effect of iCP-NI on lung histology in LPS/Poly I:C induced pneumonitis model;

FIG. 20 shows an effect of iCP-NI on the extent of alveolar injury;

FIG. 21 shows an effect of iCP-NI on a survival rate in CLP induced peritonitis model;

FIG. 22 shows an effect of iCP-NI on a survival rate in CS induced peritonitis model;

FIG. 23 shows an effect of iCP-NI on lung histology in LPS inhalation-induced acute pneumonitis model;

FIG. 24 shows an effect of iCP-NI on lung histology in Poly I:C inhalation-induced acute pneumonitis model;

FIG. 25 shows an effect of iCP-NI on the number of cells from BALF in LPS inhalation-induced acute pneumonitis model;

FIG. 26 shows pro-inflammatory cytokines (IL-12, TNF-α, IL-6 and MCP-1) suppression effect of iCP-NI from lung tissue in Poly I:C inhalation-induced acute pneumonitis model;

FIG. 27 shows pro-inflammatory cytokines (TNF-α, IL-6, IL-1β) suppression effect of iCP-NI in LPS/Poly I:C-stimulated RAW264.7 cells;

FIG. 28 shows an effect of iCP-NI on neutrophil infiltration and neutrophil counts in liver and spleen in Poly I:C/SEB systemic inflammation model;

FIG. 29 shows an effect of iCP-NI on alveolar volume in the lungs in Poly I:C/SEB systemic inflammation model;

FIG. 30 shows an effect of iCP-NI on apoptotic splenocytes;

FIG. 31 shows an effect of iCP-NI on mouse BLM induced model of pulmonary fibrosis by micro-CT;

FIG. 32 shows an effect of iCP-NI on mouse BLM induced model of pulmonary fibrosis;

FIG. 33 shows an effect of iCP-NI on O₂ saturation, respiratory rate, heart rate and body temperature of SARS-CoV-2 infected monkey model;

FIG. 34 shows an effect of iCP-NI on the level of IFN-γ in blood plasma and MCP-1 in Balf;

FIG. 35 shows an effect of iCP-NI on the viral titer of SARS-CoV-2 infected monkey model; and

FIG. 36 shows an effect of iCP-NI on immune cell infiltration, hyperplasia, hemorrhage and fibroplasia in the lungs of SARS-CoV-2 infected monkey model.

### BEST MODE FOR CARRYING OUT THE INVENTION

Hereinafter, an improved cell-permeable nuclear import inhibitor synthetic peptide for inhibition of cytokine storm or an inflammatory disease, the improved cell-permeable nuclear import inhibitor synthetic peptide including an NF-κB nuclear localization sequence and an advanced macromolecule transduction domain, a pharmaceutical composition for preventing or treating cytokine storm or an inflammatory disease, the pharmaceutical composition including the improved cell-permeable nuclear import inhibitor synthetic peptide, and a method of preventing or treating cytokine storm or an inflammatory disease according to specific embodiments of the present disclosure will be described in detail. However, these are merely an exemplary embodiment for illustration, and the scope of the present disclosure is not limited thereto, and it is apparent to those skilled in the art that various modifications to the embodiments are possible within the scope of the present disclosure.

The term “include” or “comprise” means that it includes a particular component (or element) without particular limitations unless otherwise mentioned throughout the present disclosure, and it cannot be interpreted as excluding the addition of the other components (or element).

As used herein, the term “amino acid” includes, in its broadest sense, naturally occurring L α-amino acids or residues thereof as well as D-amino acids and chemically modified amino acids. For example, the amino acid may include mimetics and analogs of the above-described amino acids. In the present disclosure, the mimetics and analogs may include functional equivalents.

As used herein, the term “inflammation” is generally a result of a localized protective response of body tissues against host invasion by foreign substances or harmful stimuli. The cause of such inflammation may include infectious causes such as bacteria, viruses, and parasites, physical causes such as burns or radiation, or chemicals such as toxins, drugs, or industrial reagents, or immune responses such as allergies and autoimmune reactions, or abnormal conditions related to oxidative stress.

As used herein, the term “preventing” means all of the actions by which the occurrence of cytokine storm or inflammatory disease is restrained or retarded by administering the improved cell-permeable nuclear import inhibitor synthetic peptide according to the present disclosure, and the term “treating” means all of the actions by which symptoms of cytokine storm or inflammatory disease have taken a turn for the better or been modified favorably by administering the improved cell-permeable nuclear import inhibitor synthetic peptide according to the present disclosure.

As used herein, the term “administering” means providing the predetermined pharmaceutical composition of the present disclosure for a subject in any appropriate way.

As used herein, the term “subject” means all animals including humans who have developed or are likely to develop cytokine storm or an inflammatory disease. The animals may include not only humans, but also cattle, horses, sheep, pigs, goats, camels, antelopes, dogs or cats in need of treatment of similar symptoms, but are not limited thereto.

According to a first embodiment, the present disclosure provides an improved cell-permeable nuclear import inhibitor (iCP-NI) synthetic peptide for inhibition of cytokine storm or an inflammatory disease, the improved cell-permeable nuclear import inhibitor synthetic peptide including an NF-κB nuclear localization sequence (NLS) and an advanced macromolecule transduction domain (aMTD).

With regard to the improved cell-permeable nuclear import inhibitor synthetic peptide of the present disclosure, the NF-κB nuclear localization sequence may be a linear NF-κB nuclear localization sequence and circular NLS with two additional cysteine. With regard to the improved cell-permeable nuclear import inhibitor synthetic peptide of the present disclosure, the circular NF-κB nuclear localization sequence may include an amino acid sequence of SEQ ID NO: 1.

With regard to the improved cell-permeable nuclear import inhibitor synthetic peptide of the present disclosure, the advanced macromolecule transduction domain may include an amino acid sequence selected from the group consisting of SEQ ID NOs: 2 to 6.

With regard to the improved cell-permeable nuclear import inhibitor synthetic peptide of the present disclosure, the improved cell-permeable nuclear import inhibitor synthetic peptide may inhibit nuclear transport of stress-responsive transcription factors (SRTFs). For example, the stress-responsive transcription factor may be NF-κB (nuclear factor KB), NFAT (nuclear factor of activated T cells), AP1 (activator protein 1), STAT1 (signal transducer and activator of transcription 1), or Nrf2 (nuclear factor erythroid 2-related factor 2).

With regard to the improved cell-permeable nuclear import inhibitor synthetic peptide of the present disclosure, the cytokine storm may cause an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease. For example, the autoimmune disease may include rheumatoid arthritis, psoriasis, atopic dermatitis, Crohn's disease, inflammatory bowel disease, Sjorgen's syndrome, optic neuritis, chronic obstructive pulmonary disease, asthma, type I diabetes, neuromyelitis optica, Myasthenia Gavis, uveitis, Guillain-Barre syndrome, psoriatic arthritis, Gaves' disease and allergy, but is not limited thereto.

According to a second embodiment, the present disclosure provides a pharmaceutical composition for preventing or treating cytokine storm or an inflammatory disease, the pharmaceutical composition including the improved cell-permeable nuclear import inhibitor (iCP-NI) synthetic peptide including the NF-κB nuclear localization sequence (NLS) and the advanced macromolecule transduction domain (aMTD).

With regard to the pharmaceutical composition for preventing or treating cytokine storm or an inflammatory disease according to the present disclosure, the NF-κB nuclear localization sequence may be a linear NF-κB nuclear localization sequence and circular NLS with two additional cysteine.

With regard to the pharmaceutical composition for preventing or treating cytokine storm or an inflammatory disease according to the present disclosure, the circular NF-κB nuclear localization sequence may include the amino acid sequence of SEQ ID NO: 1.

With regard to the pharmaceutical composition for preventing or treating cytokine storm or an inflammatory disease according to the present disclosure, the advanced macromolecule transduction domain may include an amino acid sequence selected from the group consisting of SEQ ID NOs: 2 to 6.

With regard to the pharmaceutical composition for preventing or treating cytokine storm or inflammatory diseases according to the present disclosure, the improved cell-permeable nuclear import inhibitor synthetic peptide may inhibit nuclear transport of stress-responsive transcription factors (SRTFs). For example, the stress-responsive transcription factor may be NF-κB (nuclear factor KB), NFAT (nuclear factor of activated T cells), AP1 (activator protein 1), STAT1 (signal transducer and activator of transcription 1), or Nrf2 (nuclear factor erythroid 2-related factor 2).

With regard to the pharmaceutical composition for preventing or treating cytokine storm or an inflammatory disease according to the present disclosure, the cytokine storm or inflammatory disease may be induced by inflammatory infections caused by viruses, bacteria, fungi, or parasites. The inflammatory infections may be caused by viruses, bacteria, fungi, or parasites. For example, the viruses may include coronavirus, influenza virus, Hantavirus, flavivirus, Epstein-Ban virus, human immunodeficiency virus, Ebola virus, retrovirus, or variola virus, but is not limited thereto. The coronavirus may be severe acute respiratory syndrome coronavirus 2 (SARS-CoV-2). The bacterial infection may include bacteremia, bacterial sepsis, pneumonia, cellulitis, meningitis, erysipelas, infective endocarditis, necrotizing fasciitis, prostatitis, pseudomembranous colitis, pyelonephritis, or septic arthritis, but is not limited thereto. The bacterial infection may be caused by Francisella tularensis, Streptococcus spp., Staphylococcus spp., Salmonella spp., Pseudomonas spp., Clostridium spp., Vibrio spp., Mycobacterium spp., or Haemophilus spp., but is not limited thereto. The fungi may include Aspergillis, Candida albicans, or Cryptococcus neoformans, but is not limited thereto. The parasites may include malaria parasite such as Plasmodium falciparum, but is not limited thereto.

With regard to the pharmaceutical composition for preventing or treating cytokine storm or an inflammatory disease according to the present disclosure, the cytokine storm or inflammatory disease may be induced by trauma, injury, burns, toxins, or carcinogens. The toxins may include lipopolysaccharide-induced toxins, superantigen-induced toxins (e.g., Staphylococcal enterotoxin A or B, streptococcal pyrogenic toxin and M protein, or any superantigen produced by bacteria), plant toxins (e.g., poison ivy), nickel, latex, environmental toxins (e.g., poisons) or allergies, but are not limited thereto.

With regard to the pharmaceutical composition for preventing or treating cytokine storm or an inflammatory disease according to the present disclosure, the cytokine storm may cause inflammatory diseases, for example, an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease. For example, the autoimmune disease may include rheumatoid arthritis, psoriasis, atopic dermatitis, Crohn's disease, inflammatory bowel disease, Sjorgen's syndrome, optic neuritis, chronic obstructive pulmonary disease, asthma, type I diabetes, neuromyelitis optica, Myasthenia Gavis, uveitis, Guillain-Barre syndrome, psoriatic arthritis, Gaves' disease and allergy, but is not limited thereto. With regard to the pharmaceutical composition for preventing or treating cytokine storm or an inflammatory disease according to the present disclosure, the pharmaceutical composition may further include an antibiotic, anti-viral agent (For example, remdesivir), anti-HIV agent, anti-parasite agent, anti-protozoal agent, steroidal agent, steroidal or non-steroidal anti-inflammatory agent, antihistamine (For example, diphenhydramine), immunosuppressant agent, or a combination thereof. For example, the antibiotic may include cephalosporin series, beta-lactam series, beta-lactam/beta-lactamase inhibitor series, quinolone series, glycopeptide series, carbapenem series, aminoglycoside series, macrolide series, sulfa drug series, aztreonam, clindamycin, tigecycline, colistin sodium methanesulfonate, metronidazole, spiramycin, or a combination thereof, but is not limited thereto. The cephalosporin series antibiotic may include cefazolin, cefcapene pivoxil, cefpodoxime proxetil, cephradine, ceftriaxone, cefbuperazone, cefotaxime, cefminox, ceftazidime, cefpirome, cefixime, cephalexin, cefdinir, cefroxadine, cefuroxime, cefadroxil, cefoxitin, cefetamet pivoxil, ceftizoxime, cefamandole nafate, cefazedone, cefteram pivoxil, ceftezole, cefprozil, cefotetan, cefmenoxime, cefditoren pivoxil, cefatrizine proplyene glycol, cefotiam, cefotiam hexetil HCl, ceftibuten, cefaclor, cefoperazone, cefpiramide, cephalothin, cefodizime, cefonicid, cefmetazole, or cefepime. The beta-lactam series antibiotic may include nafcillin, piperacillin, or ampicillin. The beta-lactamase inhibitor series antibiotic may include sulbactam, tazobactam, sultamicillintosylate, amoxicillin, potassium clavulanate, ticarcillin, or pivoxil sulbactam. The quinolone series antibiotic may include ciprofloxacin, moxifloxacin, levofloxacin, or lomefloxacin. The glycopeptide series antibiotic may include vancomycin, linezolid, or teicoplanin. The carbapenem series antibiotic may include meropenem, doripenem monohydrate, cilastatin, or imipenem monohydrate. The aminoglycoside series antibiotic may include amikacin, tobramycin, netilmicin, sisomicin, isepamicin, fosfomycin, or gentamicin. The macrolide series antibiotic may include clarithromycin, roxithromycin, or azithromycin. The sulfa drug series antibiotic may include sulfamethoxazole or trimethoprim.

The pharmaceutical composition of the present disclosure may further include an appropriate carrier, excipient, or diluent commonly used. The carrier, excipient, or diluent which may be included in the pharmaceutical composition of the present disclosure may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate or mineral oil, but is not limited thereto.

The pharmaceutical composition of the present disclosure may be administered in an oral or parenteral formulation according to a common method, and when formulated, commonly used diluents or excipients, such as fillers, extenders, binders, wetting agents, disintegrants, surfactants, etc., may be used. Solid formulations for oral administration include tablets, pills, powders, granules, capsules, etc., and such solid formulations are prepared by mixing the composition with at least one excipient, for example, starch, calcium carbonate, sucrose, lactose, gelatin, etc. In addition to simple excipients, lubricants such as magnesium stearate and talc are also used. Liquid formulations for oral administration include suspensions, liquid solutions for internal use, emulsions, syrups, etc. In addition to water and liquid paraffin, which are simple diluents commonly used, various excipients, such as wetting agents, sweetening agents, fragrances, preservatives, etc., may be included. Formulations for parenteral administration include sterile aqueous solutions, non-aqueous solvents, suspensions, emulsions, lyophilized formulations, and suppositories. As the non-aqueous solvents and suspensions, propylene glycol, polyethylene glycol, vegetable oil such as olive oil, and injectable ester such as ethyl oleate may be used. As a base for suppositories, witepsol, macrogol, tween 61, cacao butter, laurin butter, or glycerogelatin may be used, but are not limited thereto.

A preferred administration dosage of the pharmaceutical composition of the present disclosure may vary depending on an individual's conditions and body weight, severity of the disease, the type of drug, administration route and duration, but it may be appropriately selected by those skilled in the art. For a desirable effect, the pharmaceutical composition of the present disclosure may be administered at a dose of 0.001 mg/kg to 1000 mg/kg per day. Administration may be performed once a day, or may be divided several times. The above dosage does not limit the scope of the present disclosure in any aspect.

According to a third embodiment, the present disclosure provides a method of preventing or treating cytokine storm or an inflammatory disease, the method including the step of administering, to a subject, the improved cell-permeable nuclear import inhibitor synthetic peptide including the NF-κB nuclear localization sequence (NLS) and the advanced macromolecule transduction domain (aMTD).

With regard to the method of preventing or treating cytokine storm or an inflammatory disease according to the present disclosure, the NF-κB nuclear localization sequence may be a linear NF-κB nuclear localization sequence and circular NLS with two additional cysteine.

With regard to the method of preventing or treating cytokine storm or an inflammatory disease according to the present disclosure, the circular NF-κB nuclear localization sequence may include the amino acid sequence of SEQ ID NO: 1.

With regard to the method of preventing or treating cytokine storm or an inflammatory disease according to the present disclosure, the advanced macromolecule transduction domain may include an amino acid sequence selected from the group consisting of SEQ ID NOs: 2 to 6.

With regard to the method of preventing or treating cytokine storm or an inflammatory disease according to the present disclosure, the improved cell-permeable nuclear import inhibitor synthetic peptide may inhibit nuclear transport of stress-responsive transcription factors (SRTFs). The stress-responsive transcription factor may be NF-κB (nuclear factor KB), NFAT (nuclear factor of activated T cells), AP1 (activator protein 1), STAT1 (signal transducer and activator of transcription 1), or Nrf2 (nuclear factor erythroid 2-related factor 2).

With regard to the method of preventing or treating cytokine storm or an inflammatory disease according to the present disclosure, the improved cell-permeable nuclear import inhibitor synthetic peptide may be co-administered with an antibiotic, anti-viral agent (For example, remdesivir), anti-HIV agent, anti-parasite agent, anti-protozoal agent, steroidal agent, steroidal or non-steroidal anti-inflammatory agent, antihistamine (For example, diphenhydramine), immunosuppressant agent, or a combination thereof. For example, the antibiotic may include cephalosporin series, beta-lactam series, beta-lactam/beta-lactamase inhibitor series, quinolone series, glycopeptide series, carbapenem series, aminoglycoside series, macrolide series, sulfa drug series, aztreonam, clindamycin, tigecycline, colistin sodium methanesulfonate, metronidazole, spiramycin, or a combination thereof, but is not limited thereto.

With regard to the method of preventing or treating cytokine storm or an inflammatory disease according to the present disclosure, the administration may include intravenous, parenteral, transdermal, subcutaneous, intramuscular, intracranial, intraorbital, intraocular, intraventricular, intracapsular, intrathecal, intracisternal, intraperitoneal, intranasal, intrarectal, intravaginal, spraying, or oral administration.

With regard to the method of preventing or treating cytokine storm or an inflammatory disease according to the present disclosure, the cytokine storm may be induced from inflammatory infections caused by viruses, bacteria, fungi, or parasites.

With regard to the method of preventing or treating cytokine storm or an inflammatory disease according to the present disclosure, the cytokine storm or inflammatory disease may be induced by trauma, injury, burns, toxins, or carcinogens.

With regard to the method of preventing or treating cytokine storm or an inflammatory disease according to the present disclosure, the inflammatory disease may include an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease. For example, the autoimmune disease may include rheumatoid arthritis, psoriasis, atopic dermatitis, Crohn's disease, inflammatory bowel disease, Sjorgen's syndrome, optic neuritis, chronic obstructive pulmonary disease, asthma, type I diabetes, neuromyelitis optica, Myasthenia Gavis, uveitis, Guillain-Barre syndrome, psoriatic arthritis, Gaves' disease and allergy, but is not limited thereto.

### MODE FOR THE INVENTION

Hereinafter, the present disclosure will be described in more detail with reference to Examples and Experimental Examples. However, the following Examples and Experimental Examples are only for illustrating the present disclosure, and the content of the present disclosure is not limited to the following Examples and Experimental Examples.

### Examples

1. Stability Verification Using Reversed-Phase High-Pressure Liquid Chromatography (RP-HPLC)

The stability of iCP-NI was verified using reversed-phase high-pressure liquid chromatography (RP-HPLC). Absorbance of iCP-NI stored in two varying conditions were measured and compared: 1) stored at 25° C. for 3 months as lyophilized powder and 2) stored at −20° C. as lyophilized powder. 1 mg of iCP-NI lyophilized powder from each condition was dissolved in 1 mL of HPLC-grade water and the solution was filtered with 0.2 μm pore syringe filter (ADVANTEC, Cat No. 13HP020AN). 50 μL of iCP-NI were injected into a reversed-phase-column (25 cm×4.6 mm, HRC-ODS; SHIMADZU, Cat No. 228-23463-92). The conditions were: buffer A; 0.05% Trifluoroacetic acid (TFA) in distilled water and buffer B; 0.05% TFA in acetonitrile. The chromatographic run was performed by applying a gradient of 0-100% of buffer B for the course of 60 min at a flow rate of 1 mL/min at 35° C. The peaks were monitored by measuring the absorbance at 230 nm. The chromatograms were derived and analyzed using Agilent Open LAB Control Panel Software.

2. Binding Affinity Measurement Using Surface Plasmon Resonance (SPR)

Surface Plasmon Resonance (SPR) was performed to verify direct interaction between iCP-NI and importin a5. Biacore T200 apparatus from Cytiva was used to carry our SPR. Temperature was fixed at 25° C. for all experiments. Running buffer was prepared in double-distilled water (DW) as followed: 25 mM HEPES, 100 mM NaCl, 0.05% surfactant P20, pH 7.5. The flow system was primed three times before initiating an experiment. The CM5 sensor chip (Cytiva, BR100530, 10285242) was used in all experiments. The sensor chip surface was rinsed with three injections of running buffer before importin a5 immobilization. Importin a5 was injected into the CM5 sensor chip to achieve the Rmax value of 1900 RU. The diluted concentration (between 10, 25, 50, 75 and 100 μM) of iCP-NI was injected onto the sensor chip at a rate of 30 μL/min for a total of 180 s. CM5 sensor chip surface regeneration was performed with 100 mM NaOH (30 μL/min for 60 s). Baseline response values were compared before and after each experiment to evaluate the effectiveness of the surface regeneration. After the affinity measurement between the iCP-NI and importin a5 was completed, the dissociation constant (KD) was calculated using Biacore T200 Evaluation Software 3.1.

3. Pharmacokinetic Analysis and Distribution of iCP-NI

To evaluate the pharmacokinetics of iCP-NI in the lungs, 6-8 weeks old C57BL/6 mice were intravenously administered with 100 mg/kg of FITC-conjugated iCP-NI. Subsequently, mice were sacrificed at various time points and lung tissues were obtained for pharmacokinetic analysis. Also, other tissues (brain, heart, liver, spleen, kidney, small/large intestine and pancreas) were collected for distribution of iCP-NI analysis. Representative lung tissue specimens were perfusion-fixed with 4% paraformaldehyde (PFA; BIOSESANG, Cat No. PC2031-100-00) and incubated in 30% sucrose solution overnight at 4° C. Next, specimens were embedded in optimal cutting temperature (OCT) compound (Leica Biosystems, Cat No. 3801480) and cryo-sectioned to a 10 μm thickness onto microscope glass slide. The cryo-sectioned samples were washed thoroughly with PBS and incubated with ammonium chloride (NH4Cl, Sigma-Aldrich, Cat No. A9434) solution for 20 minutes at room temperature (RT). The samples were mounted with DAPI-containing mounting solution (Invitrogen, Cat No. 00-4959-52) and visualized by fluorescence microscopy using a confocal laser scanning microscope system (Leica SP8) and the images were processed with Leica LAS X software).

4. Cell Culture System

Murine macrophage cell line (RAW264.7), murine dendritic cell line (DC2.4), human monocyte cell line (THP-1) and human alveolar epithelial cell line (A549) were purchased from Korean Cell Line Bank (KCLB) and human T lymphocyte (Jurkat T cell, E6-1) was purchased from American Tissue Culture Collection (ATCC). RAW264.7 cells were maintained in Dulbecco's modified Eagle's medium (DMEM; Hyclone, Cat No. SH30243.01) containing 10% fetal bovine serum (FBS; Hyclone, Cat No. SH30919.03) and 1% penicillin/streptomycin (Hyclone, Cat No. SV30010). DC2.4, THP-1, Jurkat T and A549 cells were cultured in RPMI-1640 medium (Hyclone, Cat No. SH30255.01) containing 10% FBS and 1% penicillin/streptomycin. All cells were maintained at 37° C. in a humidified atmosphere with 5% CO2 incubator.

5. Isolation of Mouse Bone Marrow-Derived Macrophages (BMDMs)

Bone marrow-derived macrophages (BMDMs) were isolated from the bone-marrow (BM) of 8-weeks old C57BL/6J mice. BM cells were obtained from femurs and tibia of mice and flushed out with serum-free DMEM media. The single cell suspension was then filtered through a nylon cell strainer (70-μm Nylon mesh) and washed twice with HBSS media. Cells were centrifuged (200×g, 5 minutes) and the supernatant was aspirated. The cells were lysed with Red Blood Cell Lysis Buffer (Sigma-Aldrich, Cat No. R7757) for 3 minutes at RT and reaction was blocked by diluting the lysis buffer with serum-containing DMEM media. Cells were centrifuged (200×g, 5 minutes) and the supernatant was discarded. The cells were cultured in 12-well tissue culture plates (1×10⁶ cells/well) with complete BMDM medium [DMEM containing 10% FBS, 1% penicillin-streptomycin supplemented with 50 ng/mL of M-CSF (Peprotech, Cat No. 315-02)] and maintained at 37° C. and 5% CO2 in a humid incubator. On days 1 and 3, the medium was replaced, and on day 5 differentiated BMDMs were adhered and used in experiments.

6. Cell Stimulation, Activation and Transfection

All cells were stimulated using LPS (Sigma-Aldrich, Cat No. L3012), human IFN-γ (R&D Systems, Cat No. 285-IF), mouse IFN-γ (R&D Systems, Cat No. 485-MI-100), phorbol myristate acetate (PMA; Sigma-Aldrich, Cat No. P1585), ionomycin (Sigma-Aldrich, Cat No. 10634) and Poly I:C (Sigma-Aldrich, Cat No. P1530). The activation protocol and activation times points for each experiment are described in the individual figure legends. For Poly I:C intracellular delivery, RAW264.7 cells were transfected with Poly I:C using lipofectamine 3000 (Thermo Scientific, Cat No. L3000-015), according to the manufacturer's instructions.

7. Animal Systems

Male C57BL/6 mice (6-8 weeks old) were purchased from Nara-Biotech and Samtako, all animal experiments were performed in compliance with the institutional recommendations in the National Guidelines for the Care and Use of Laboratory Animals. All protocols were approved by the Institutional Animal Care and Use Committee (IACUC; Approval No. CV-2019-32) of the Institute of Laboratory Animal Resources at Cellivery.

8. LPS/D-Galactosamine-Induced Acute Liver Injury Model

LPS and D-galactosamine (Sigma-Aldrich, Cat No. G0500) were used for acute liver injury mice model. Male C57BL/6 (6-weeks old) mice were administered PBS (100 μL/mouse) or iCP-NI (50 mg/kg, 100 μL/mouse) by intravenous (I.V) injection. After 1 hours, LPS (50 μg/kg in 200 μL) and D-galactosamine (400 mg/kg in 200 μL) were intraperitoneally injected into individual mice. LPS/D-galactosamine induced mice were injected PBS (100 μL/mouse) or iCP-NI (100 μL/mouse) via I.V. at each time points. The specific protocol as described in the individual figure legends. All animals were monitored and sacrificed at 16 hours after LPS/D-gal challenge.

9. Toxin Inhalation-Induced Acute Pneumonitis Model

Male C57BL/6 (6-8 weeks old) mice were administrated PBS (100 μL/mouse) or iCP-NI (50 mg/kg, 100 μL/mouse) by I.V injection. PBS or iCP-NI injected mice were administered with LPS or Poly I:C through inhalation using a nebulizer (Omron, Cat No. NE-U22). All mice were placed in a custom-made acryl-cage for 30 minutes with inhalation of LPS (0.5 mg/mL, 7 mL) or Poly I:C (1 mg/mL, 5 mL). Next, iCP-NI were administered through tail vein injection after completion of inhalation as described in the individual figure legends.

10. IT LPS/Poly I:C Mouse Model

Male C57BL/6 mice (6-8 weeks old) were anaesthetized with isoflurane and received both LPS and Poly I:C via intratracheal administration. Normal animals received 50 μL of PBS alone. LPS dissolved in PBS and 10 mg/kg of LPS (50 μL/mouse) was administered. After 4 hours, LPS-sensitized mice were anaesthetized with isoflurane and 50 mg/kg of Poly I:C (100 μL/mouse) was injected intratracheally. LPS/Poly I:C-challenged mice were randomly assigned to two different groups. The treatment groups were as follows: (1) I.T LPS/Poly I:C, PBS, 38 animals (2) I.T LPS/Poly I:C, iCP-NI, 25 animals. Mice were treated with PBS (100 μL/mouse) or iCP-NI (30 mg/kg, 100 μL/mouse) administered by I.V injection at 4, 16, 28 and 52 hours after Poly I:C injection.

11. Cecal Slurry-Induced Peritonitis Model

Polymicrobial sepsis was studied using a cecal slurry (CS)-induced peritonitis model (1-3). 8-weeks old male C57BL/6 mice were euthanized, sacrificed, and the whole cecum was separated for dissection in each mouse. The entire cecal contents were collected and mixed with 4 mL of PBS to create CS with a concentration of 1 g/mL. This CS was filtered through sterile 70 μm strainers and mixed with an equal volume of 30% glycerol in PBS to produce a final stock solution (lx CS) in 15% glycerol. Final solution was then aliquoted and stored under −80° C. For induction of mouse sepsis, frozen CS stock (1.5 mg/kg) was thawed under 37° C. and peritoneally injected into mouse model using 1 mL syringe. 25 mg/kg of meropenem (Sigma-Aldrich, Cat No. 1392454) was first administered 2 hours after CS injection, followed by a regular administration every 24 hours. iCP-NI (50 mg/kg) was first injected 2 hours following the cecal slurry administration and was given 4 times with 2-hour interval. Both meropenem and iCP-NI were injected intravenously.

12. Cecal-Ligation and Puncture (CLP)-Induced Peritonitis Model

Cecal-ligation and puncture (CLP)-induced peritonitis models are considered the most suitable for the purpose of the study due their tendency to develop sepsis similar to that off human, which results in systemic bacteremia, organ dysfunction and eventual systemic inflammation (4-6). Male C57BL/6 mice (8-weeks old) were anesthetized with alfaxan (Jurox, Cat No. 470760) and rompun (Bayer) mixture (7:3) applied via intramuscular injection and placed in supine position. After shaving and performing aseptic preparations, 1-2 cm midline incision was made through the abdominal wall. The cecum was partially ligated with a 6-0 silk suture. The puncture of the cecal wall was performed with a 19-gauge needle and cecal contents were gently leaked. The incision was closed with 6-0 silk suture autoclip. Povidone-iodine was applied to sutured area afterwards to avoid infection of the surgical site.

13. Pneumonitis-Induced Systemic Inflammation Model (Poly I:C/SEB)

Poly I:C and streptococcal enterotoxin B (SEB; Sigma-Aldrich, Cat No. BT202) used for the pneumonitis-induced systemic inflammatory model. Male C57BL/6 (6-weeks old) mice were administrated PBS (100 μL/mouse) or iCP-NI (50 mg/kg, 100 μL/mouse) by I.V injection. PBS or iCP-NI injected mice were inhaled Poly I:C (1 mg/mL, 5 mL) for 30 minutes using a nebulizer. After then, mice were anesthetized with mixture of alfaxan and rompun (7:3) via intramuscular injection and administered SEB (0.25 mg/kg, 20 μL/mouse) via intranasal route. Poly I:C/SEB-induced mice were injected PBS (100 μL/mouse) or iCP-NI (50 mg/kg, 100 μL/mouse) via I.V. at each time points. The specific protocol as described in the individual figure legends. All animals were sacrificed at 0.5, 1, 2, 4 or 6 hours after Poly I:C/SEB challenge.

14. Bleomycin-Induced Pulmonary Fibrosis Model

Bleomycin was used to develop experimental model for lung fibrosis (7, 8). Bleomycin sulphate (BLM; Sigma-Aldrich, Cat No. B2434) was dissolved in sterile PBS and used in experiment. 8-weeks old male C57BL/6 mice were anesthetized with isoflurane and received either PBS or bleomycin via intratracheally on Day 0. Single intratracheal (I.T) injections of bleomycin (3 mg/kg in 50 μL PBS) were administered to animals (n=41) mice. Normal animals (n=10) received 50 μL of PBS alone. Bleomycin received mice were randomly assigned to two different groups. The treatment groups were as follows: (1) I.T bleomycin, PBS, 21 animals (2) I.T bleomycin, iCP-NI, 20 animals. Mice were treated with PBS (100 μL/mouse) or iCP-NI (50 mg/kg, twice a day, 100 μL/mouse) administered by intravenous (I.V) injection for 7 days. For micro-computed tomography (CT) scanning, mice were anaesthetized with isoflurane and the mouse lung imaging was taken using In Vivo X-ray Radiography Micro-CT System. Mice were sacrificed and lung tissue were collected for histopathological analysis.

15. Study of SARS-CoV-2-Infected Non-Human Primates (NHPs)

15-1. SARS-CoV-2-Infected Non-Human Primates (NHPs) Models

The study was designed to assess the therapeutic efficacy of iCP-NI against SARSCoV-2 in rhesus macaques (RM) and African green monkeys (AGM). Animals were housed in Southern Research's (SR) A/BSL-3 facility during the study period. Virus strain (2019-nCoV/USA-WA1/2020) was originated from CDC and was provided by UTMB Galveston. Animals were anesthetized and a feeding tube was inserted into the trachea. Once the end of the tube had reached the mid-point of the trachea, the animal was held in a sitting position and the inoculate (1.0 or 4.0 mL) was instilled through the feeding tube followed by a sufficient amount of flush material (sterile DPBS without Mg2+/Ca2+) to ensure complete delivery of the challenge material. The challenge virus was used at 4.0×106 TCID 50/mL on Day 0. Vials of SARS-CoV-2 were thawed and vortexed for 10 seconds prior to preparation of challenge and stored on ice unit dose administration. Animals were anesthetized with ketamine and xylazine for inoculation. Approximately 24 hours after challenge, the intravenous (IV)-designated animals were administered a single IV infusion of 200 mg/kg/animal of iCP NI for 30 min. The dosing material was administered by infusion pump and the blood vessel used was documented. The dose was delivered in a volume of approximately 30 mL at a rate of 1 mL per minute. All animals were monitored for clinical signs, body temperatures, glucose levels, 02 saturation, heart rate (BPM), respiration rate, and body weights. Necropsy was performed and tissues were collected for histopathology on Day 5 and 8.

15-2. Blood Collection, Processing & Cytokine Analysis.

For all animals, blood was collected for cytokine and glucose level evaluation. Approximately 6 mL was collected from anesthetized animals, 2 mL was used to process plasma (cytokine analysis) in EDTA tubes and 4 mL was used for PBMCs in sodium heparin tubes on Days −3, 2, 4, 5, 6 and 8. Glucose level was monitored from blood collected via a finger or toe prick. Blood samples were taken from any accessible vein. Blood was collected for moribund animals prior to euthanasia. Blood collected into tubes with EDTA and sodium heparin anticoagulant was gently inverted immediately following collection. Cytokine levels (IL-6, IL-10, MCP1, INF-beta, INF gamma, TNF-alpha, IL-1 beta, IL-2, IL-4, and IL-17a) were measured using Luminex.

15-3. Necropsy and Histopathology

All animals received a limited postmortem examination of the lung, liver, kidney, and spleen. Samples of the tissues were fixed in 10% neutral buffered formalin. The lung, liver, kidney, and spleen tissues were collected and weighed from all necropsied animals and fixed in 10% neutral buffered formalin for histopathology. Collected tissues were processed to slides and stained with hematoxylin and eosin (all tissues) and trichrome (lungs only).

16. Cytometric Bead Array (CBA) & Enzyme-Linked Immunosorbent Assay (ELISA)

16-1. Cytokine Estimation in Mice Plasma and Tissue

The concentration of TNF-α, IL-6, IL-12, IL-10 and IFN-γ was measured in blood plasma samples, bronchoalveolar lavage fluid (BALF) or lung tissue using cytometric bead array (BD Biosciences, Cat. No 552364), following manufacturer's protocol. Blood plasma was collected from LPS/D-Galactosamine-induced acute liver injury mice, LPS or Poly I:C inhalation-induced pneumonitis mice. Bronchoalveolar lavage fluid (BALF) was collected from LPS or Poly I:C inhalation-induced pneumonitis mice. Lung tissue homogenates obtained from LPS/Poly I:C induced pneumonitis mice. Data were acquired in a LSRII flow cytometer (BD Biosciences) and analyzed with FCAP Array software (BD Biosciences).

16-2. Enzyme-Linked Immunosorbent Assay

Cytokines contained in supernatants from the cell culture were analyzed for TNF-α, IL-6, IL-1β content in duplicate using a commercially available ELISA kit for mouse TNF-α (Invitrogen, Cat No. 88-7324-88), mouse IL-6 (Invitrogen, Cat No. 88-7064-88) and mouse IL-1β (Invitrogen, Cat No. 88-7013-88). The assay was performed according to the manufacturer's protocol.

17. Cytoplasmic/Nucleus Extract Preparation

To analyze nuclear translocation inhibition of stress response transcription factors (SRTFs) in immune cells, the cytoplasmic and nuclear extraction was prepared using an NE-PER Nuclear Cytoplasmic Extraction Reagent kit (Thermo Scientific, Cat No. 78835) according to the manufacturer's instruction. Briefly, the stimulated cells were washed twice with ice-cold PBS and centrifuged at 500×g for 3 minutes. Supernatant were removed carefully and the cell pellet was suspended in cytoplasmic extraction reagent I (CER I) supplemented with protease/phosphatase inhibitors by vortexing. The suspension was incubated on ice for 10 minutes followed by the addition of cytoplasmic extraction reagent II (CER II), vortexed for 5 seconds, incubated on ice for 1 min and centrifuged at 16,000×g for 5 minutes. The supernatant (cytoplasmic extract) was transferred to a pre-chilled tube. The insoluble pellet fraction, which contains nuclei, was resuspended in nuclear extraction reagent (NER) by vortexing during 15 seconds every 10 minutes and incubated on ice for 40 minutes, then centrifuged for 10 min at 16,000×g. The supernatant, constituting the nuclear extract, was used for the subsequent experiments.

18. Western Blot Analysis

Total cell lysates and cytoplasmic/nucleus protein used in experiment. Total cell pellets were lysed in RIPA buffer (Biosesang, Cat No. RC2002-050-00) supplemented with protease/phosphatase inhibitor (Cell Signaling, #5872S), and lysates were collected. Protein samples were quantified using Bradford assay (Bio-Rad). Samples containing 30 μg of protein were mixed with SDS-sample buffer and boiled for 5 minutes. Protein was separated by 10% SDS-polyacrylamide gel electrophoresis and transferred onto polyvinylidene fluoride (PVDF; Merck Millipore, Cat No. IPVH00010) membranes. The blotted membranes were blocked in 5% Bovine Serum Albumin (BSA; BIOSESANG, Cat No. A1025) in Tris-buffered saline containing 0.1% Tween-20 (Biopure, Cat No. TWN508-500) for 1 hour at RT and then incubated with primary antibodies at 4° C. overnight followed by incubation with horseradish peroxidase (HRP)-conjugated secondary antibodies for 1 hour at RT. The protein bands were visualized by ChemiDoc imaging system (Bio-Rad) using chemiluminescent reagents (Super Signal™ West Dura Extended Duration Substrate; Thermo Scientific, Cat No. 34075) and quantified with Image J software. Antibodies used were as follows: anti-phospho-NF-κB p65 Ser536 (Cell Signaling, #3033), anti-NF-κB p65 (Santa Cruz, sc-8008), anti-NFATc1 (Santa Cruz, sc-7294), anti-phospho-c-Jun Ser63 (Cell Signaling, #91952), anti-c-Jun (Santa Cruz, sc-74543), anti-phospho-STAT3 Tyr705 (Cell Signaling, #9145), anti-STAT3 (Cell Signaling, #9139), antiphospho-STAT1 Tyr701 (Cell Signaling, #9167), anti-STAT1 (Santa Cruz, sc-464), anti-IL-1β (R&D Systems, AF-401-NA), anti-snail (Cell Signaling, #3879), anti-slug (Cell Signaling, #9585), anti-twist (Santa Cruz, sc-81417), 13-Actin (Sigma-Aldrich, Cat No. A3854), anti-LaminB1 (Santa Cruz, sc-374015), anti-GAPDH (Bioworld, Cat No. MB001H), horseradish peroxidase (HRP)-conjugated anti-rabbit IgG antibody (Cell Signaling, #7074) and horseradish peroxidase (HRP)-conjugated anti-mouse IgG (Cell Signaling, #7076).

19. RNA Extraction and Quantitative RT-PCR

Total RNA was isolated from cells or tissue using TRIzol reagent (Thermo Scientific, Cat No. 15596018), and cDNA was synthesized from total of 1 μg of RNA using a iScript cDNA synthesis kit (Bio-Rad, Cat No. 1708897) in accordance with the manufacturer's instructions. qPCR was conducted with iQ SYBR® Green Supermix (Bio-Rad, Cat No. 1708880) on the Bio-Rad CFX96 Real-Time PCR Detection system (Bio-Rad) with the following steps: (1) polymerase and DNA denaturation, 95° C. for 5 min, (2) denaturation, 95° C. for 10 sec, (3) annealing and extension 60° C. for 30 sec for 35 cycles, 4) melt curve analysis, 60 to 95° C. increment, 5 sec/step. The relative amount of mRNA (2 ^{ΔΔ}Ct) was obtained by normalizing its level to that of the β-actin gene.

20. Flow cytometry for cell permeability Analysis

RAW264.7 cells (5×10⁵ cells/well) were seeded in 12-well and incubated for 24 hours. The cells were washed twice with PBS and treated with FITC-conjugated iCP-NI, FITC-conjugated-cNLS or FITC peptide control in FBS-free medium for 1 hour at 37° C. or 4. Then cell supernatants were removed, the cell pellets were collected and washed three times with ice-cold PBS. Next, the cells were fixed using 70% EtOH for 30 minutes at 4° C. and washed three times with ico-cold PBS. Internalized peptides of the cells were analyzed using LSRII flow cytometry system (BD).

To evaluate the cellular uptake of the peptides, RAW264.7 cells were treated with different types of agents related to the intracellular mechanism. ATP (Sigma-Aldrich, Cat No. A2383)-depleting agent (Antimycin; Sigma-Aldrich, Cat No. A8674), proteinase K (Cosmogenetech, Cat No. CMB-022), microtubule inhibitor (Taxol; Sigma-Aldrich, Cat No. T7402), clathrin-mediated endocytosis blocker (chlorpromazine; Sigma-Aldrich, Cat No. C8138), lipid raft-mediated endocytosis blocker (methyl-β-cyclodextrin; Sigma-Aldrich, Cat No. C4555) or macropinocytosis blocker (amiloride; Sigma-Aldrich, Cat No. A7410) were used for analysis. The procedure was as follows: RAW264.7 cells were pre-treated with (1) 10 μM of antimycin in the presence or absence of 1 mM ATP for 2 hours, (2) 10 μg/ml proteinase K for 10 minutes, (3) 20 μM of Taxol, (4) 3 μM of chlorpromazine for 30 minutes, (5) 5 mM of methyl-βcyclodextrin for 30 minutes. (6) 10 μM antimycin in the presence or absence of 1 mM ATP for 2 hours in serum-free medium. After then, the cells were post-treated with FITC-conjugated iCP-NI, FITC-conjugated-cNLS or FITC peptide control. Cell fixation and analysis are as described above.

21. Immunocytochemistry (ICC)

The cells were fixed in 4% paraformaldehyde for 15 minutes at RT and permeabilized using methanol at −20° C. Next, the cells were incubated in blocking solution (3% BSA and 0.3% Triton X-100 in PBS) prior to incubation with primary antibody for 60 minutes at RT. Primary antibody was added for overnight at 4° C., and the cells were washed with PBS. Additionally, secondary antibody was added for 1 hour at RT. Next, the cells were washed with PBS and mounted onto slide with Fluoromount-GTM, with DAPI (Invitrogen, Cat No. E132139). The preparation obtained were observed and photographed using a confocal microscope equipped with a digital imaging system (Leica TCS SP8-STED). Images were processed and recorded with Leica LAS X software. Antibodies used were as follows: anti-phospho-NF-κB p65 Ser536 (Cell Signaling, #3033), anti-phospho-c-Jun Ser63 (Cell Signaling, #91952), anti-phospho-STAT1 Tyr701 (Cell Signaling, #9167), anti-phospho-STAT3 Tyr705 (Cell Signaling, #9145), NFAT (Abcam, ab2722), vimentin (Abcam, ab8978), anti-E-cadherin (Abcam, ab40772), Goat anti-Rabbit IgG Alexa Fluor 488 (Invitrogen, Cat No. A11034), Goat anti-rabbit IgG Alexa Fluor 488 (Invitrogen, Cat No. A11001), Goat anti-rabbit IgG Alexa Fluor 647 (Invitrogen, Cat No. A21245), Goat anti-mouse IgG Alexa Fluor 647 (Invitrogen, Cat No. A21235), anti-mouse CD3 PE-conjugated Antibody (R&D Systems, Cat No. FAB4841P), anti-mouse CD45R PE-conjugated Antibody (eBioscience, Cat No. 12-0452-82), anti-mouse CD11b-PE-conjugated Antibody (eBioscience, Cat No. 12-4801-82).

22. Immunohistochemistry (IHC)

All tissues were fixed with 10% buffered formalin for 24 hours and embedded in paraffin. Paraffin-embedded tissues were sectioned in thickness of 4 μm. To deparaffinization, the samples were incubated in 65° C. for 30 minutes, then incubated in fresh xylene three times for 20 minutes in each. Sample rehydration was executed in 100%, 90%, and 70% EtOH for 5 minutes, respectively. Then, samples were incubated in boiled pH 6 sodium citrate solution for 30 minutes to retrieve the antigen and transferred into NH4Cl solution for 20 minutes incubation. After washing with PBS, the samples were incubated in blocking solution (3% BSA, 0.3% Triton X-100 in PBS) was incubated for 1 hour at RT. PE-conjugated Ly6G rat monoclonal primary antibody (Thermo Scientific, Cat No. 12-9668-82) identifying neutrophil was diluted in blocking solution and incubated with samples overnight at 4° C. After rinsing with PBS, samples were mounted with mounting solution containing with DAPI. All samples were visualized by fluorescence microscopy using a confocal laser scanning microscope system (Leica TCS SP8) and the images were processed with Leica LAS X software.

23. Tissue Staining for Histopathological Analysis

All tissues were fixed with 10% buffered formalin for 48 hours and embedded in paraffin. 4-μm sections were stained with hematoxylin and eosin (H&E; Abcam, Cat No. ab245880), Masson's trichrome (Abcam, Cat No. ab150686), sirius red staining (Abcam, Cat No. ab150681) to evaluate inflammation, collagen deposition and fiber, respectively. In addition, terminal deoxynucleotidyl transferase dUTP nick terminal labeling (TUNEL) staining was performed using an Apoptag Peroxidase staining kit (Millipore, #S7101). Total specimens were observed and photographed using a microscope equipped with a digital imaging system (Nikon DS-Ri2). All experiments were conducted according to the manufacture's protocols.

24. Statistical Analysis

Statistical analyses were performed using GraphPad Prism software 5 and Microsoft Excel software. All results were performed with two biological replicates and at least of tree-independent experiments. All experiments data are shown as the means±standard deviation (SD) and a Student's t test was used to compare differences between groups. *P values of <0.05, <0.01 or <0.001 were considered statistically significant.

### Experimental Examples

1. Development of an improved cell-permeable nuclear import inhibitor, iCP-NI

We developed an improved cell-permeable nuclear import inhibitor (iCP-NI) using sequences optimized for intracellular protein delivery, designated advanced macromolecule transduction domains, or aMTDs. The aMTDs incorporated 6 critical features (amino acid length (12 amino acids), bending potential, rigidity/flexibility, structural feature, hydropathy and amino acid composition, worldwide patent, WO2016028036A1) and have significantly outperformed earlier generations of unoptimized sequences to deliver large protein cargoes such as enhanced green fluorescent protein, Parkin, and Suppressor of Cytokine Signaling 3 (SOCS3).

Peptides used in the present study are shown in Table 1. Linear NLS (1NLS) consisted of a linear nuclear localization sequence from NF-κB1 (p50) flanked by two cystines. Circular NLS (cNLS) had the same sequence circularized by a disulfide bond. 6 peptides contained the circularized NLS and different sequences to promote intracellular protein delivery: unoptimized FGF-4 sequence (cSN50.1) and 5 aMTDs (aMTD385-cNLS, aMTD666-cNLS, aMTD891-cNLS, aMTD831-cNLS, and aMTD827-cNLS). An additional peptide (aMTD827-1NLS) was identical to aMTD827-cNLS except the NLS was not circularized.

TABLE 1

| | Peptides | CPP | NLS |
|---|---|---|---|
| Structure 1 | NLS | | VQRKRQKLMP |
| | | | (SEQ ID NO. 8) |
| Structure 2 | CSN50.1 | AAVALLPAVLLA | CVQRKRQKLMPC |
| | | LLAP (SEQ ID | (SEQ ID NO. 1) |
| | | NO. 7) | |
| Structure 3 | aMTD385- | IVAIAVPALVAP | CVQRKRQKLMPC |
| | cNLS | (SEQ ID NO. 2) | (SEQ ID NO. 1) |
| Structure 4 | aMTD666- | AAIAIIAPAIVP | CVQRKRQKLMPC |
| | cNLS | (SEQ ID NO. 3) | (SEQ ID NO. 1) |
| Structure 5 | aMTD827- | IAAVLAAPALVP | CVQRKRQKLMPC |
| | cNLS | (SEQ ID NO. 4) | (SEQ ID NO. 1) |
| Structure 6 | aMTD831- | IIVAVAPAAIVP | CVQRKRQKLMPC |
| | cNLS | (SEQ ID NO. 5) | (SEQ ID NO. 1) |
| Structure 7 | aMTD891- | ILAVAAIPAALP | CVQRKRQKLMPC |
| | cNLS | (SEQ ID NO. 6) | (SEQ ID NO. 1) |
| Structure 8 | aMTD827- | IAAVLAAPALVP | CVQRKRQKLMPC |
| | 1NLS | (SEQ ID NO. 4) | (SEQ ID NO. 1) |
| Structure 9 | cNLS | | CVQRKRQKLMPC |
| | | | (SEQ ID NO. 1) |

2. Selection of Optimal iCP-NI

2-1. Anti-Inflammatory Activity of Peptides in Table 1

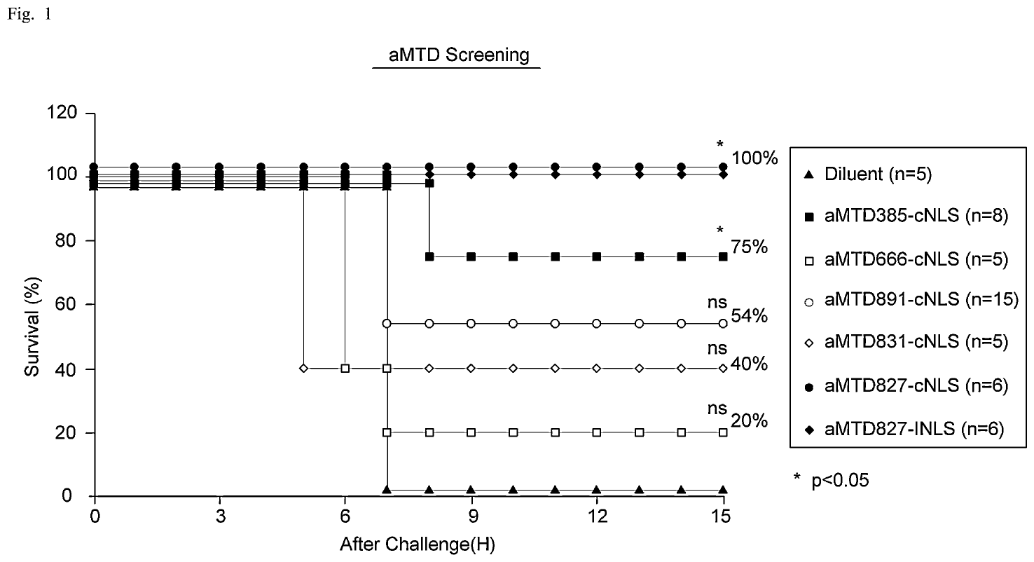

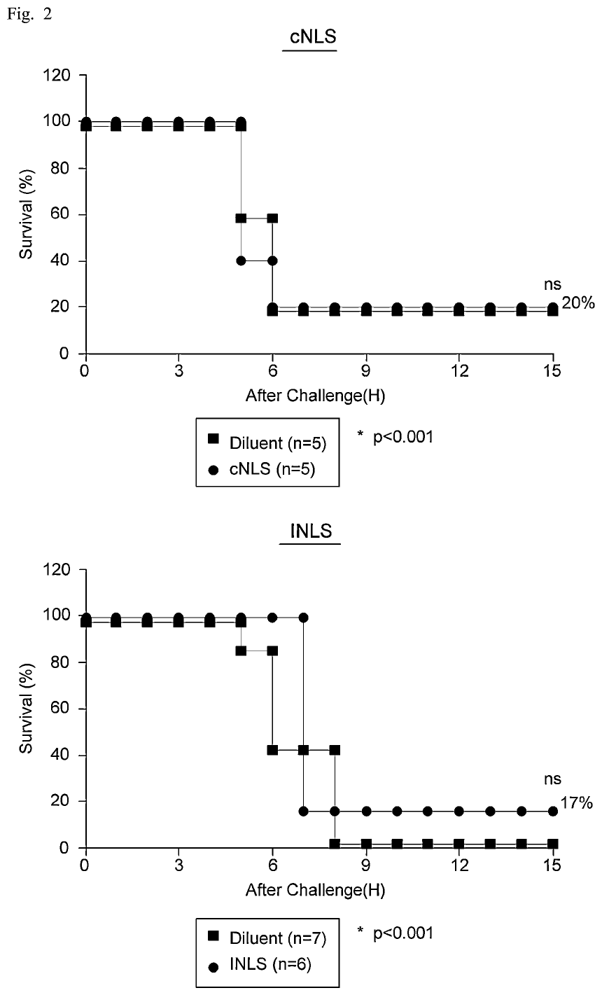

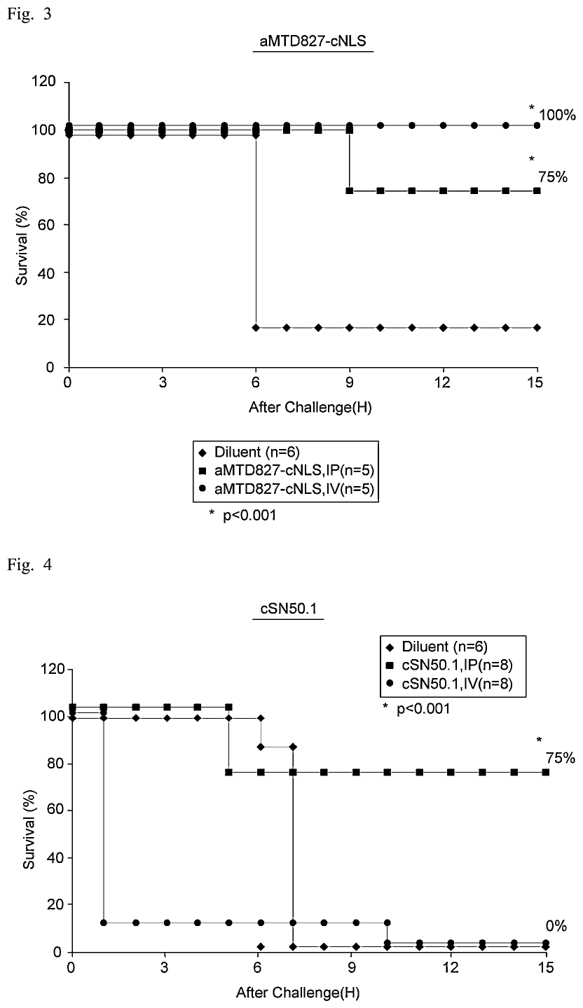

The anti-inflammatory activity of each peptide was assessed in a murine acute liver injury model in which >90% of mice succumbed to a fatal inflammatory reaction 8 hours after administration of bacterial lipopolysaccharide (LPS) and the sensitizing agent, D-galactosamine (D-Gal). Treatments with the 5 aMTD-containing peptides protected 20 to 100% of the animals tested. Circularized (aMTD827-cNLS) and linear (aMTD827-1NLS) were equally effective (FIG. 1). The aMTD827 sequence was strictly required for the anti-inflammatory activity of aMTD827-cNLS, as survival was not enhanced by treatment with the NLS sequence alone in either a linear (1NLS) or circular (cNLS) form (FIG. 2). aMTD827-cNLS was more effective when administered intravenous (IV) rather than intraperitoneal (IP) injection (100% and 75% protection, respectively, FIG. 3). The cSN50.1 peptide was less effective than aMTD827-cNLS regardless of the route of administration (FIG. 4). Based on these observations, aMTD827-cNLS was chosen as a candidate anti-inflammatory COVID-19 therapeutic.

2-2. Therapeutic Protocol of iCP-NI

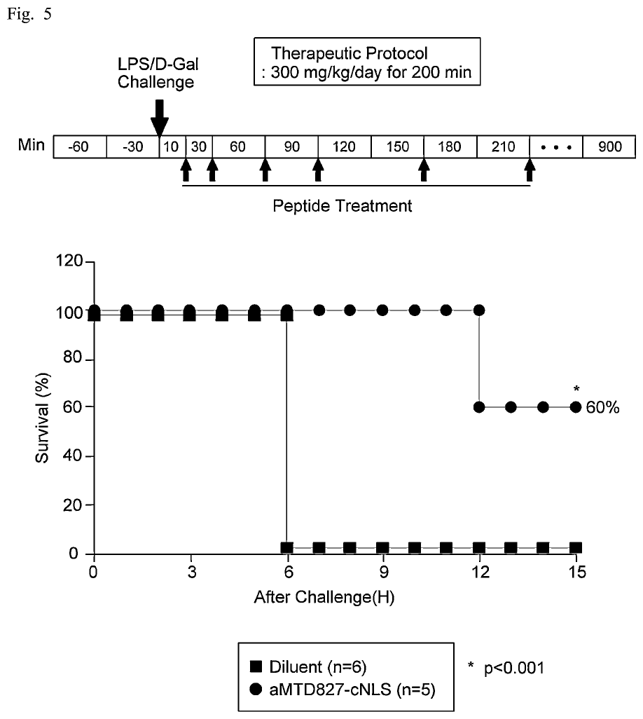

We tested several treatment regimens in the acute liver injury model and achieved 100% protection with four 50 mg/Kg treatments or with six 25 mg/Kg treatments. Regimens with four 25 mg/Kg or three 25 mg/Kg treatments were less effective, though the difference was not statistically significant. Higher levels of fatality were observed with regimens administering four 5 mg/Kg or 10 mg/Kg 40 (25% and 38% survival, respectively). Furthermore, we establish therapeutic protocol of the acute liver injury model and observed 60% therapeutic effect from six times 50 mg/kg administrated mice in this model (FIG. 5).

2-3. Stability and Ability to Bind Importin-a5 of iCP-NI

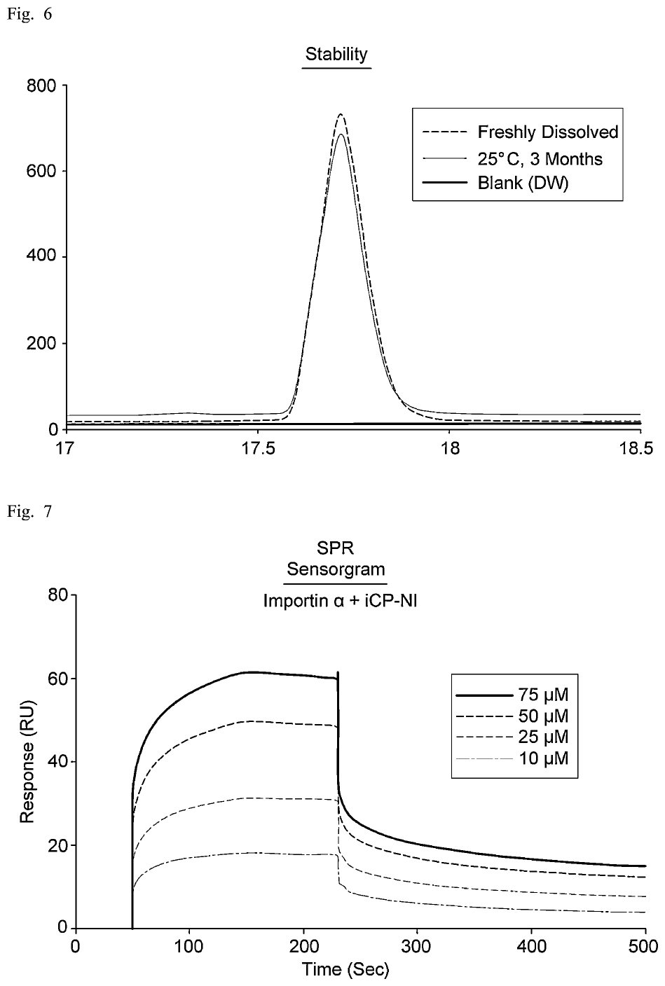

iCP-NI had excellent solubility, was stable as a lyophilized powder for over 3 months at 25° C. (FIG. 6), and retained the ability to bind Importin-a5 as assessed by surface plasmon resonance (FIG. 7).

2-4. Intracellular Delivery of iCP-NI

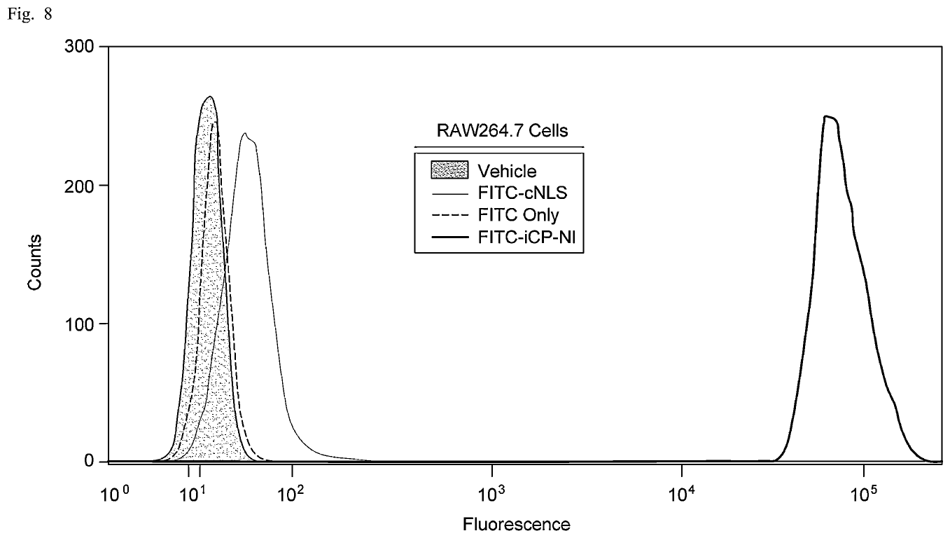

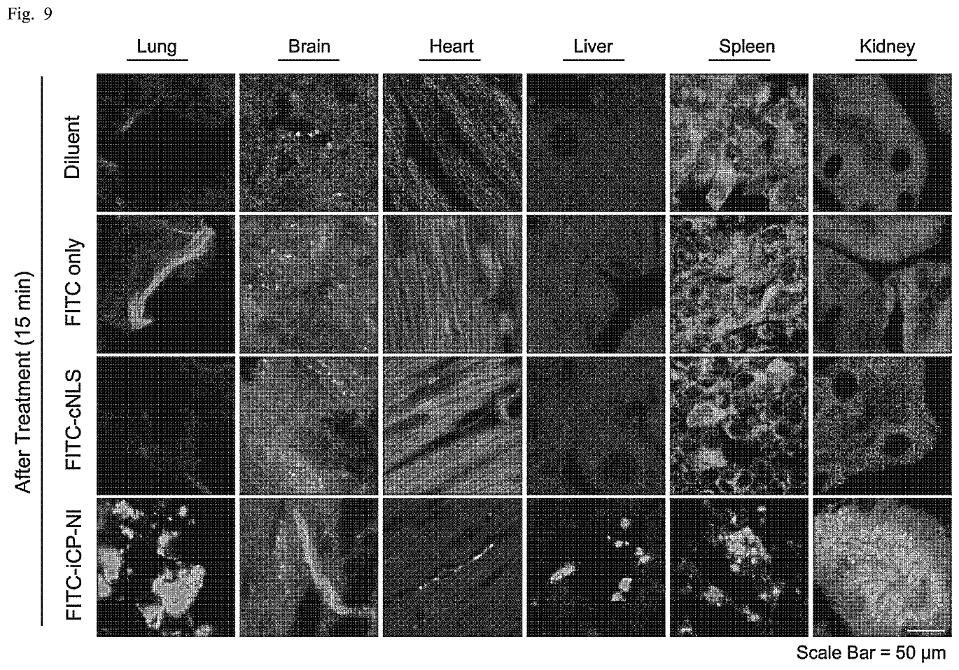

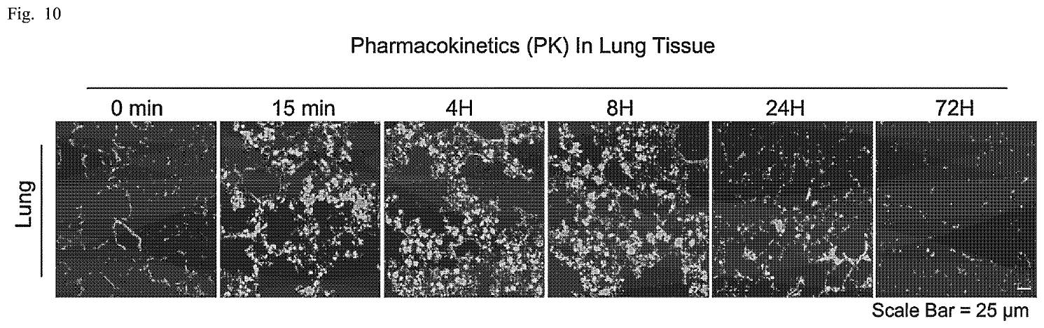

To analyze intracellular delivery, iCP-NI was labeled with fluorescein isothiocyanate (FITC) and uptake in cultured RAW264.7 cells was monitored by flow cytometry. iCP-NI displayed cell permeability 1000 times higher than the NLS without aMTD827 (FIG. 8). After IV administration, FITC-iCP-NI was widely distributed among major organs, including lungs, as well as brain, heart, liver, spleen, and kidney (FIG. 9). High levels of iCP-NI remained in lung tissue at least for 8 hours, and declined in time-dependent manner (FIG. 10).

3. Efficacy iCP-NI on Nuclear Translocation of SRTFs

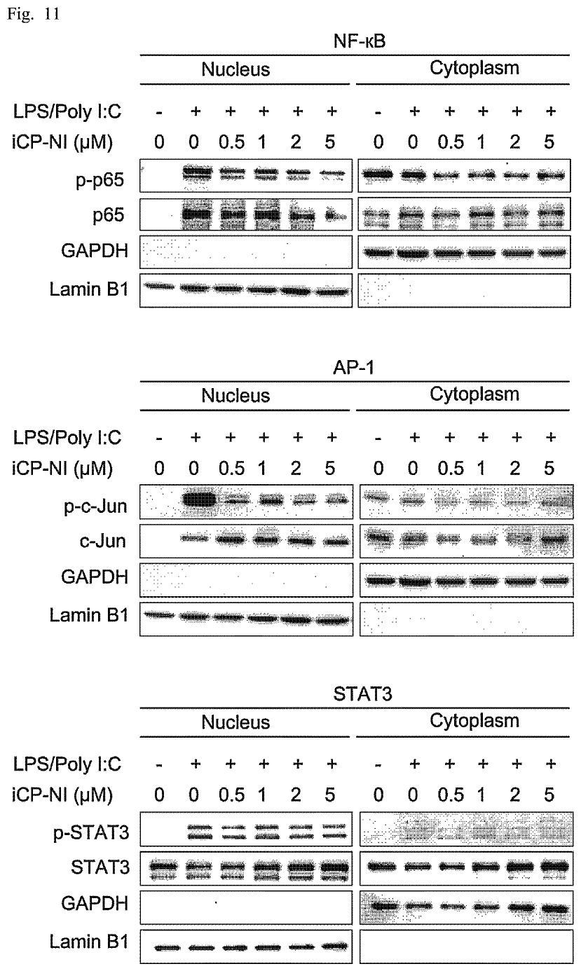

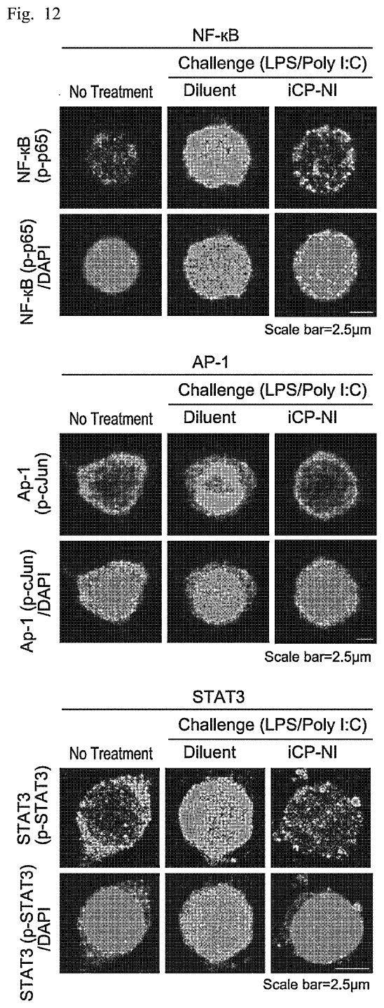

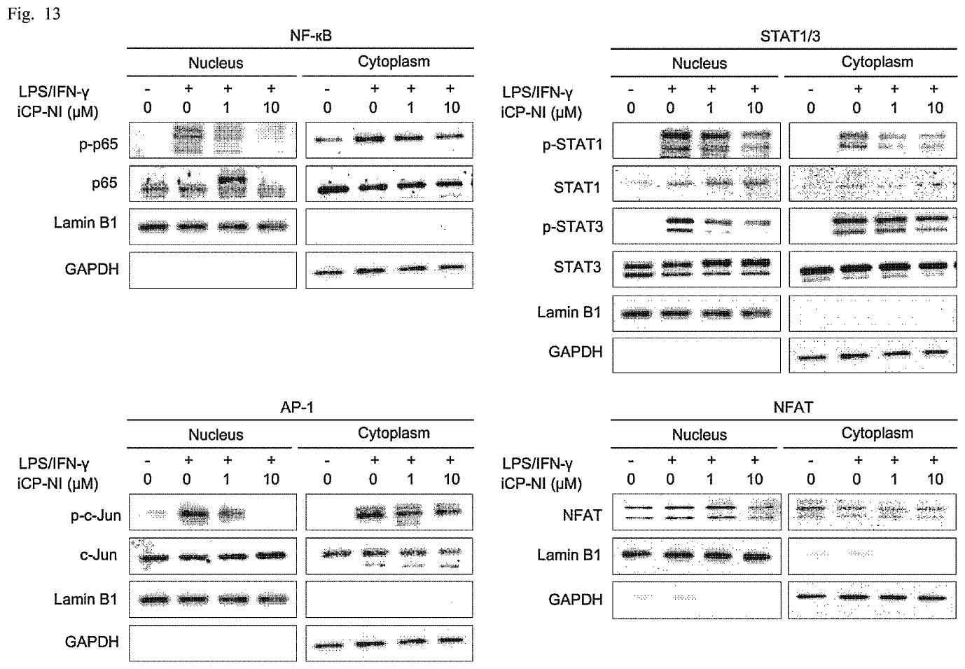

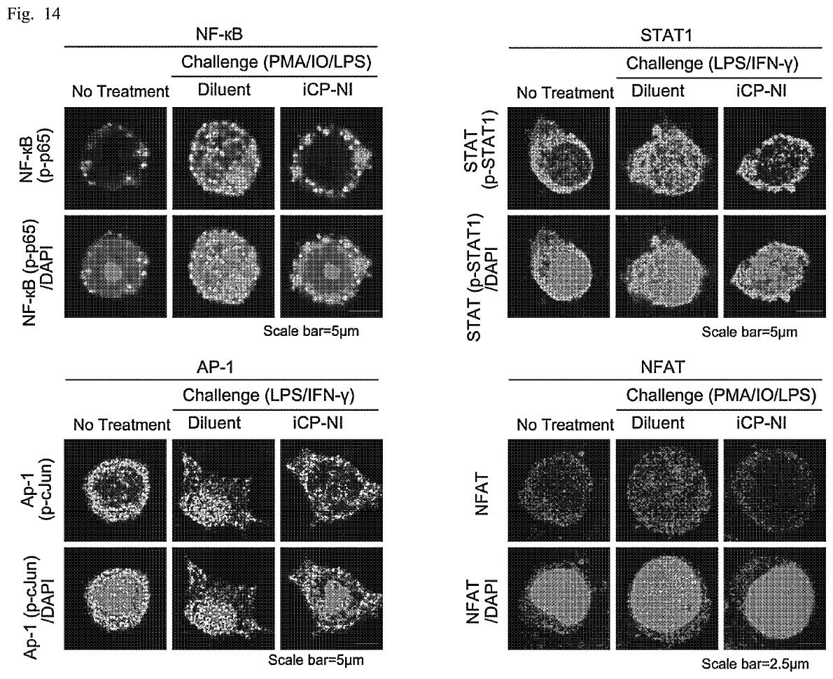

Stress-responsive transcription factors (SRTFs) activate programs of cytokine and chemokine gene expression in response to proinflammatory stimuli. Most SRTFs are regulated at the level of nuclear translocation, as illustrated by accumulations of nuclear factor kappa B (NF-κB), activator protein 1 (AP-1), signal transducer and activator of transcription 1 and 3 (STAT1 and STAT3) and nuclear factor of activated T-cells (NFAT) in the nuclei of RAW264.7 cells treated with LPS and Poly I:C or with LPS and interferon gamma (IFN-γ). iCP-NI inhibited nuclear translocation of NF-κB (phosphorylated and unphosphorylated p65) and activator protein 1 and AP-1 (phosphorylated c-Jun) and STAT3, as assessed by Western Blot analysis of nuclear and cytoplasmic fractions (FIG. 11) and by immunostaining (FIG. 12). iCP-NI also blocked nuclear accumulations of NF-κB (phosphorylated and unphosphorylated p65), STAT1 and STAT3 (phosphorylated proteins, p-STAT1 and p-STAT3), AP-1 (phosphorylated c-Jun), and NFAT in response to LPS/IFN-γ, as assessed both by Western Blot analysis (FIG. 13) and by immunostaining (FIG. 14).

4. Efficacy of iCP-NI in Acute Inflammation Models

Inhibition of SRTF nuclear translocation by iCP-NI is consistent with a model in which the NLS sequence carried by iCP-NI competes with SRTFs for binding to Importin-5a, thereby inhibiting SRTF nuclear transport. As a consequence, systemic delivery of iCP-NI is expected to suppress inflammatory disorders in which SRFTs, and the cytokines and chemokines whose expression they regulate, play significant pathological roles. This prediction was tested in several terminal mouse models of acute inflammation: LPS/D-Gal induced hepatitis, LPS/Poly I:C induced pneumonitis and peritonitis induced by cecal ligation and puncture (CLP) and cecal slurry (CS).

4-1. LPS/D-Gal Induced Hepatitis

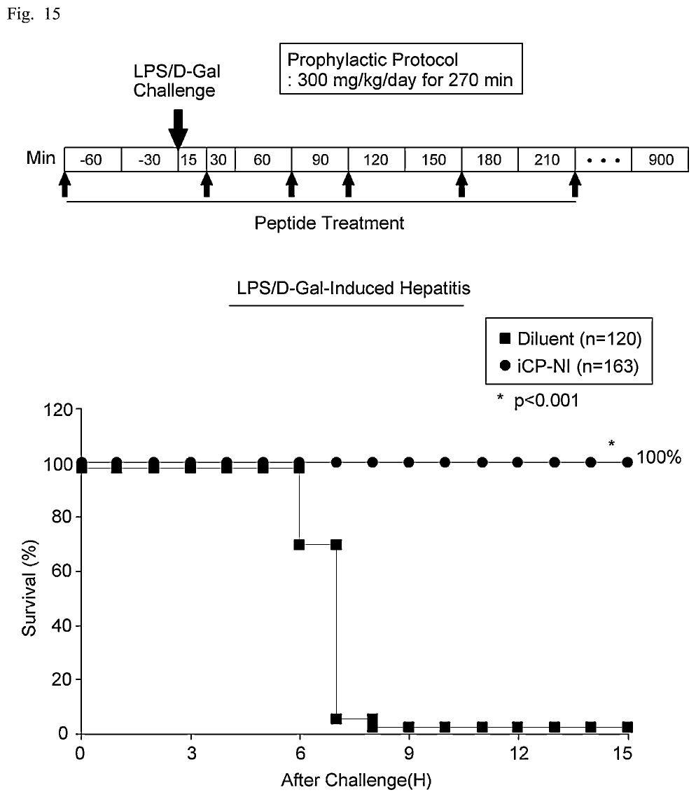

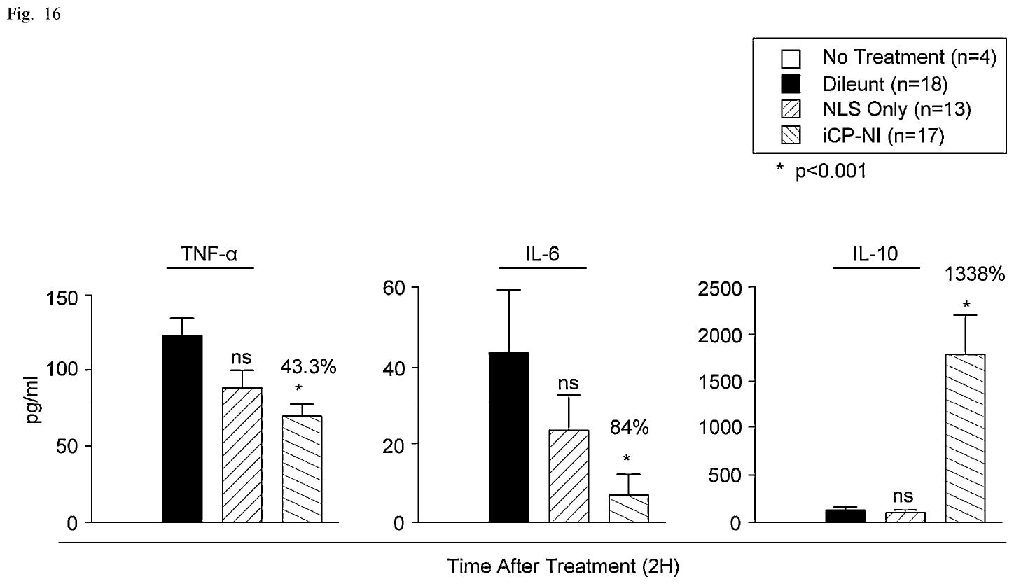

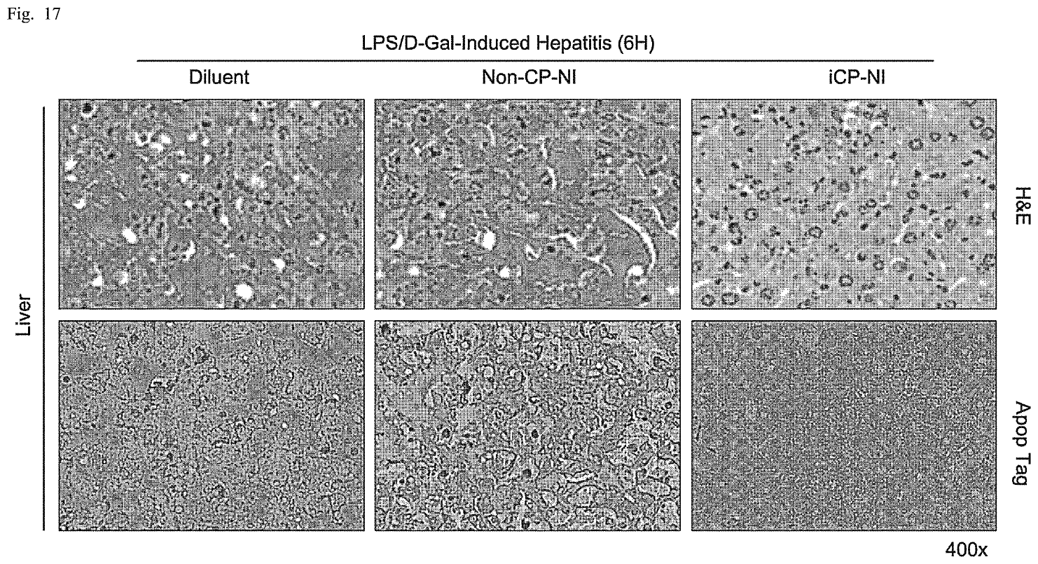

Intravenously administered iCP-NI protected mice from invariably fatal (n=120) hepatitis induced by LPS/D-Gal as evidenced by 100% survival (n=163) (FIG. 15). iCP-NI treatment suppressed the expression of inflammatory cytokines (TNF-α and IL-6) and induced IL-10 expression, an anti-inflammatory cytokine (FIG. 16) and protected against liver damage and massive liver apoptosis (FIG. 17). The striking benefit of prophylactic iCP-NI in LPS/D-Gal hepatitis model illustrates the acute consequences of SRTFs activation and accompanying inflammatory response on liver homeostasis.

4-2. LPS/Poly I:C Induced Pneumonitis

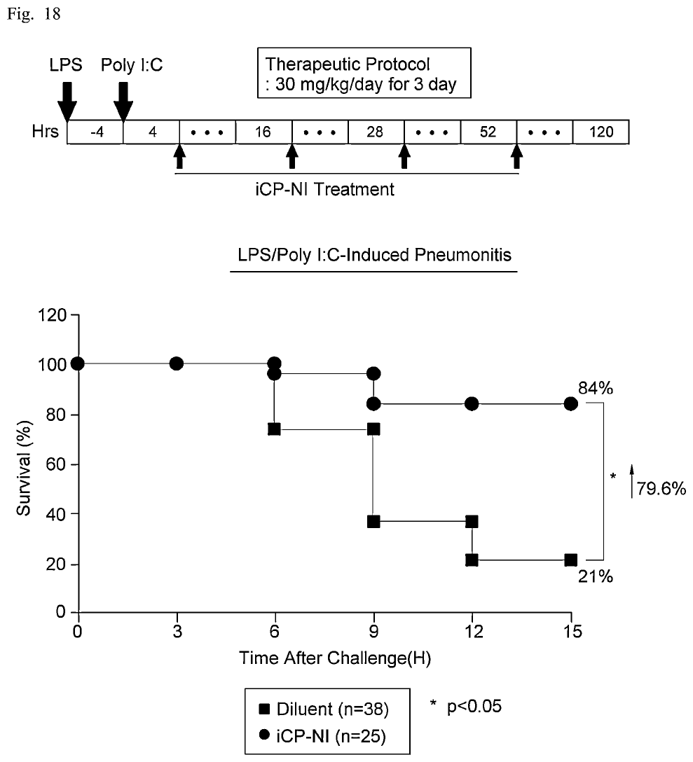

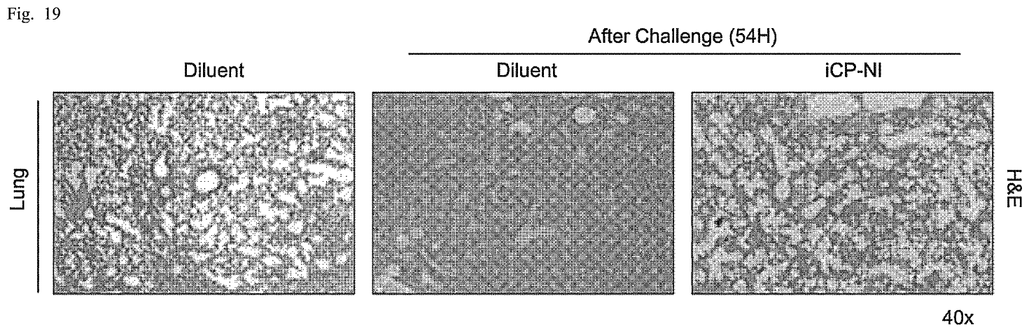

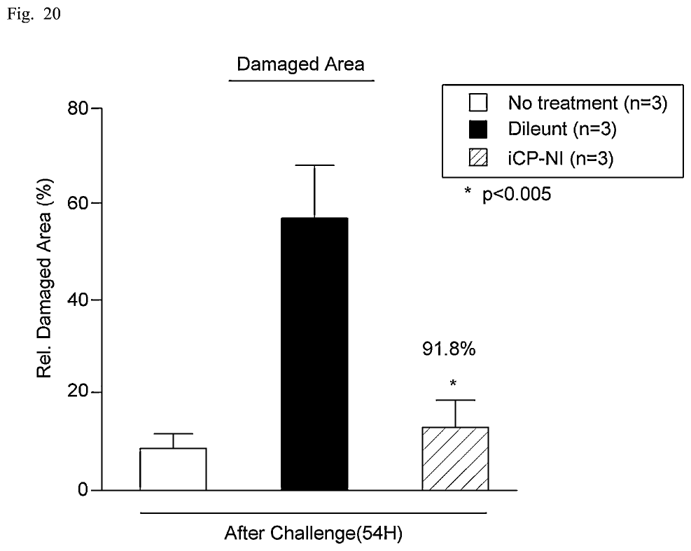

LPS was administered first followed 4 hours later by Poly I:C. Both were administered intratracheally in C57BL/6 mice. Neither treatment alone was fatal (data not shown), but the combination was fatal in 79% of animals. The mice exhibited ARDS-like symptoms including labored breathing with noises, greatly reduced activity and death within 120 hours after administering Poly I:C. By contrast treatments with iCP-NI starting 4 hours after Poly I:C increased survival to 84% (70.6% of therapeutic efficacy) (FIG. 18). Lung histology, characterized by epithelial hyperplasia, monocyte infiltration and loss of alveolar structure, was also markedly improved by iCP-NI treatments (FIG. 19), as was the extent of alveolar injury (FIG. 20).

4-3. Peritonitis Induced by Cecal Ligation and Puncture (CLP) and Cecal Slurry (CS).

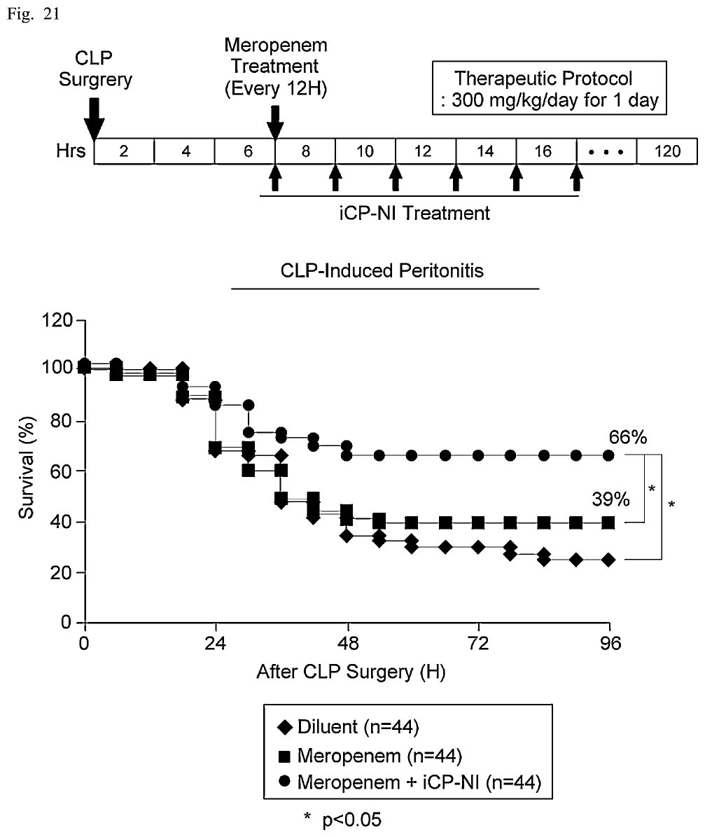

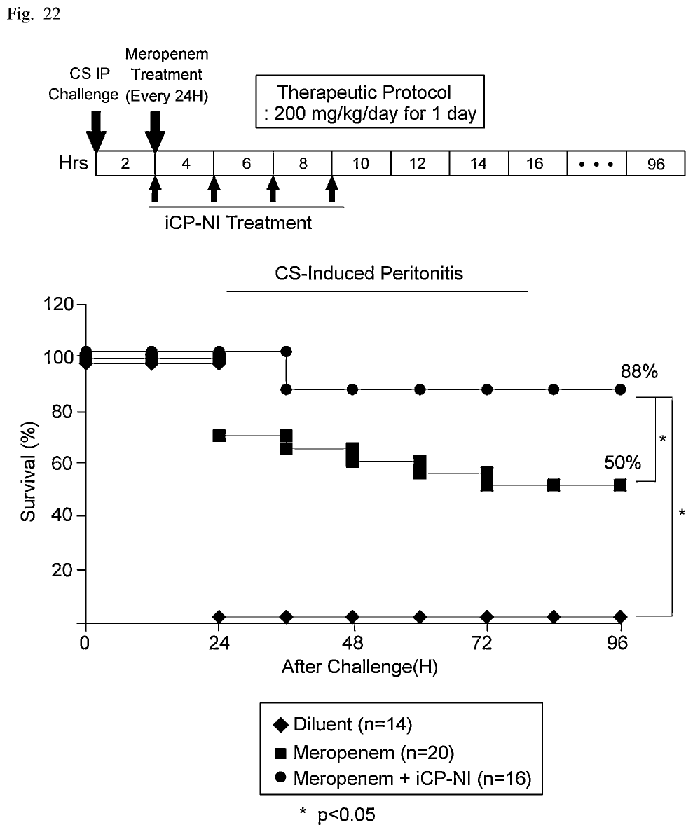

iCP-NI also proved beneficial in treating severe sepsis induced by the cecal ligation and puncture (CLP) (FIG. 21) and cecal slurry (CS) (FIG. 22) protocols. iCP-NI enhanced survival in both models beyond that achieved by the antibiotic Meropenem alone.

5. Efficacy of iCP-NI in Sub-Acute Pulmonary Inflammation Models

COVID-19 displays highly variable disease severity and progression. This is thought to result from complex interactions between proximal (respiratory tract and lungs) and distal tissues infected by the virus, the systemic effects of host inflammatory responses and patient variables such as, comorbidities, genetics and therapy, e.g. ventilator induced lung injury (VILI). The complexity of human COVID-19 complicates development of animal models and hence, the preclinical evaluation of iCP-NI as a COVID-19 therapy. We therefore took a broad approach and employed several nonlethal pneumonitis models to assess the effects of iCP-NI on a variety of pathological endpoints: lung histology, BALF cell counts and cytokine expression after LPS inhalation; lung histology, BALF cell counts and cytokine expression after poly (I:C) inhalation; lung histology, neutrophil infiltration and systemic effects in liver and spleen after poly I:C inhalation and intranasal administration of staphylococcal enterotoxin B (SEB); and bleomycin-induced lung fibrosis. In addition, we examined the effects of iCP-NI on TGF-b-induced epithelial to mesenchymal transition in A549 human alveolar epithelial cells.

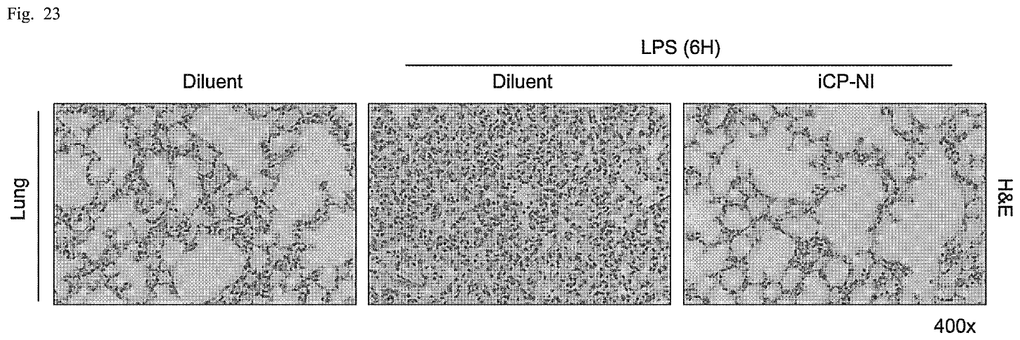

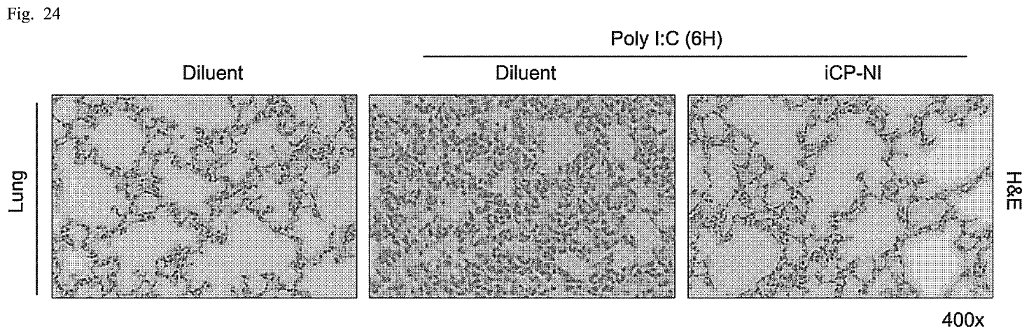

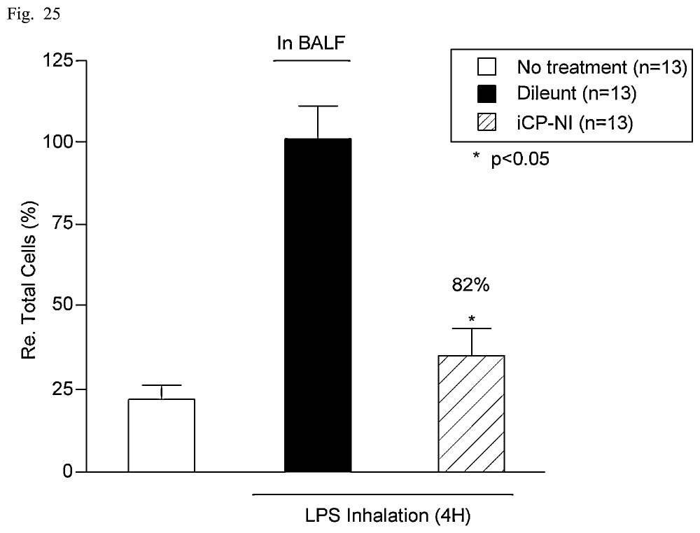

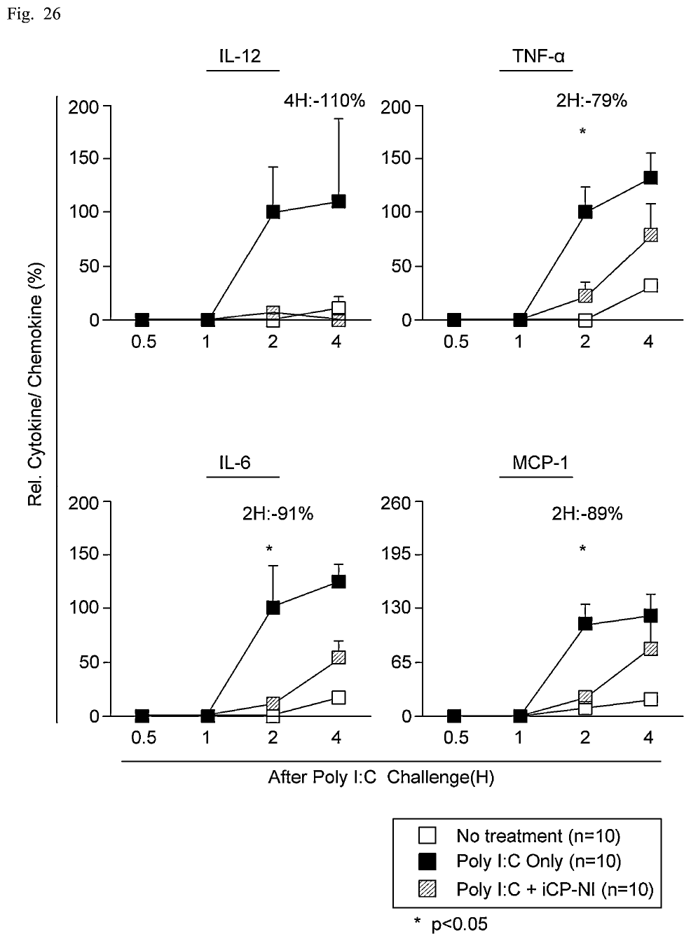

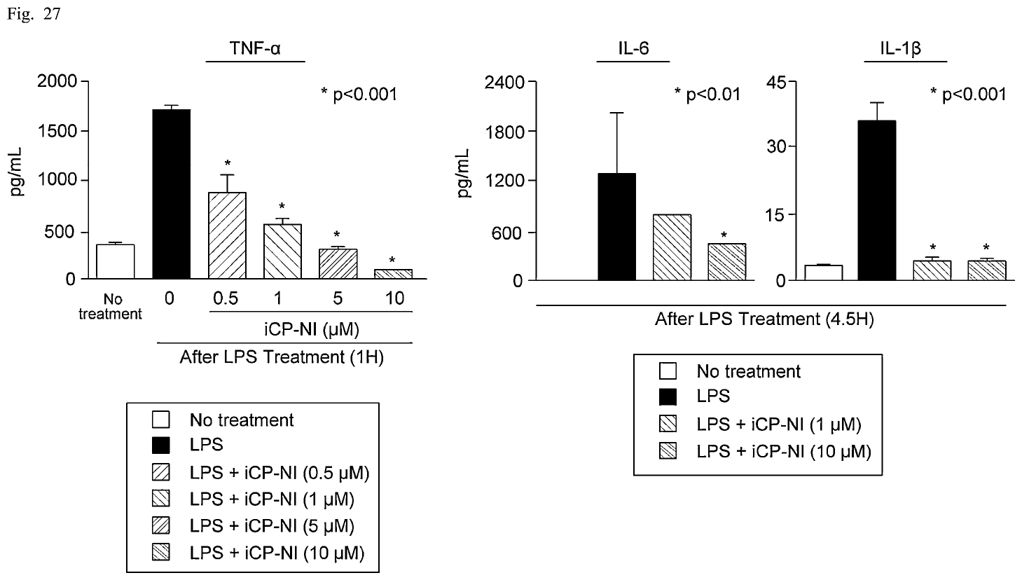

Inhaled LPS or Poly I:C induced transient changes in lung histology characterized by epithelial hyperplasia, monocyte infiltration and loss of alveolar structure. iCP-NI treatments initiated before LPS or Poly I:C blocked the greater part of these responses and preserved alveolar structure as compared to normal controls (FIGS. 23 and 24), and 5˜6 folds increases in the number of cells recovered in BALF (FIG. 25). The anti-inflammatory effects of iCP-NI were accompanied by decreases in the IL-12, TNF-α, IL-6, and MCP-1 expression (FIG. 26). Additionally, similar activation of cytokine expression by LPS was observed in RAW264.7 cells. The level of TNF-α, IL-6 and IL1β was decreased in iCP-NI treated cell in concentration-dependent of iCP-NI (FIG. 27).

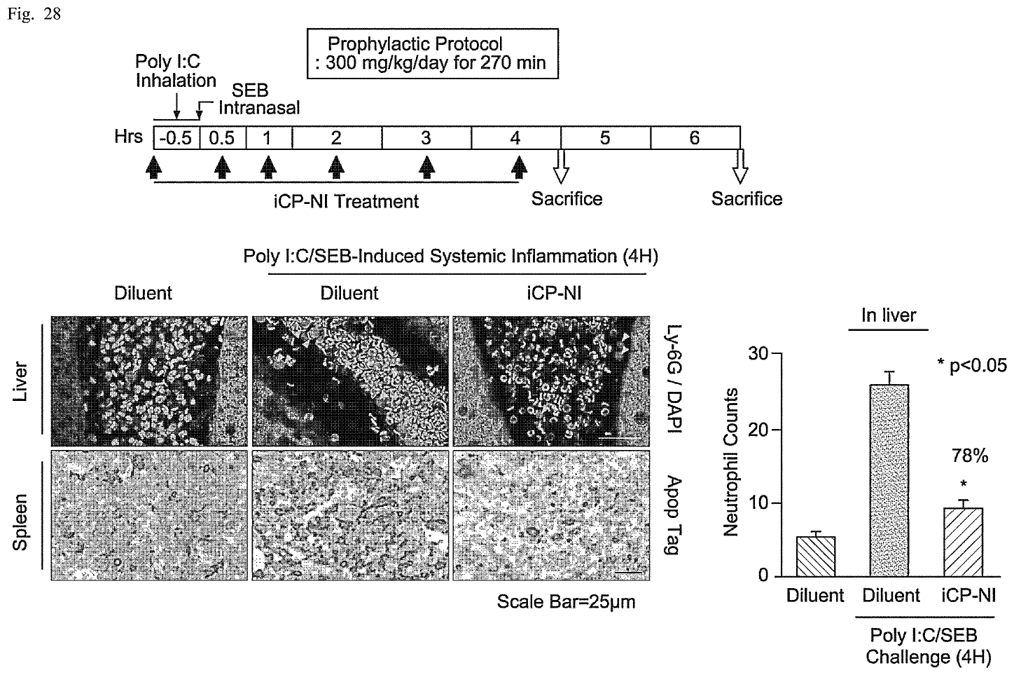

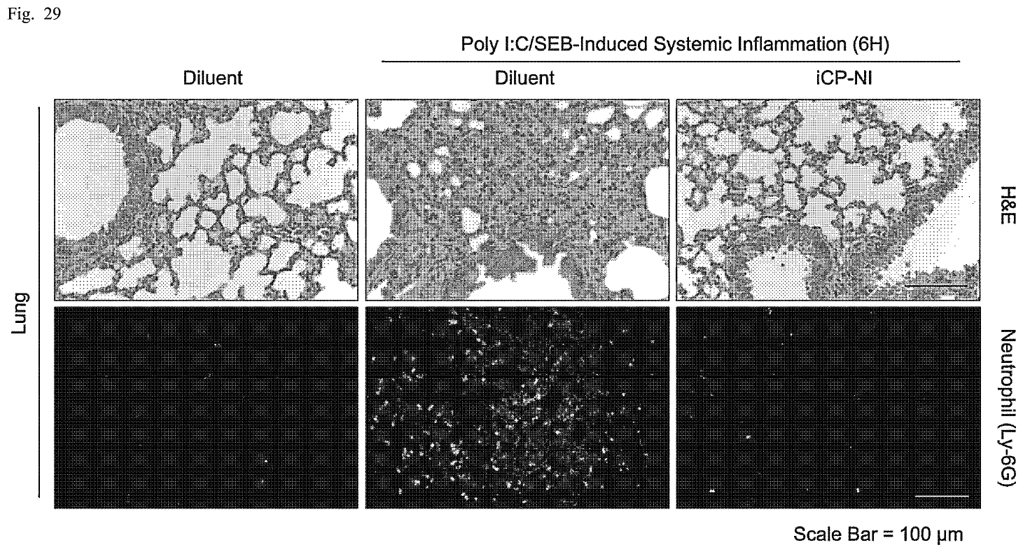

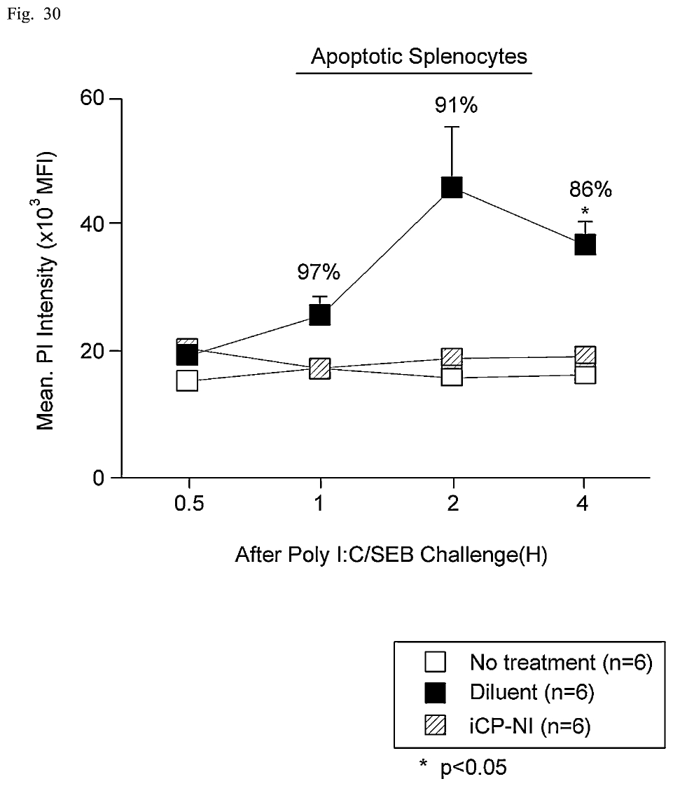

Intranasal (IN) administration of SEB after Poly I:C inhalation induced increases in cellularity, neutrophil infiltration (Ly-6G positive cells) and increased neutrophil counts that were suppressed by iCP-NI treatment. iCP-NI reduced neutrophil infiltration and counts in liver (FIG. 28) and alveolar volume in the lungs (FIG. 29). Systemic effects were also markedly improved by iCP-NI treatment, including reductions in the number of apoptotic cells-potentially a result of activation-dependent cell death-observed in the spleen (FIGS. 28 and 30).

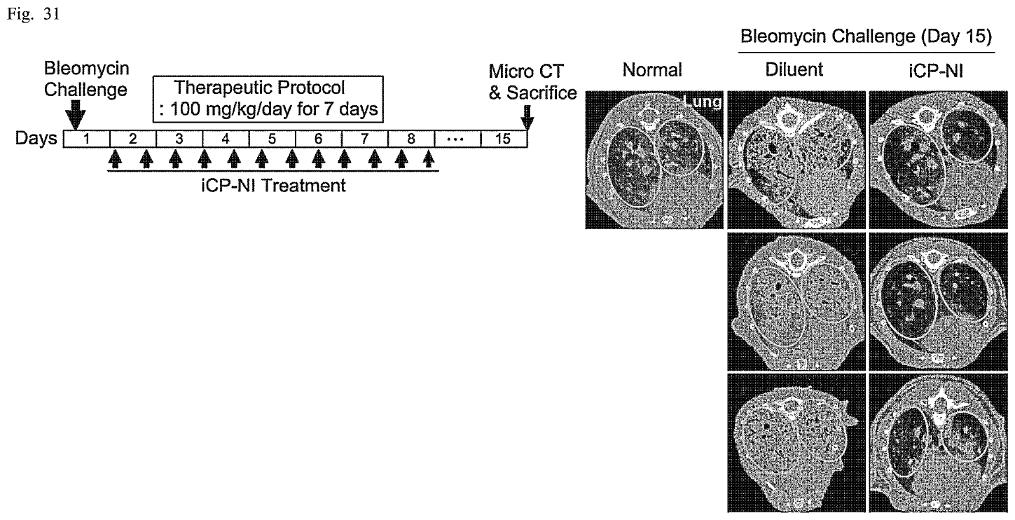

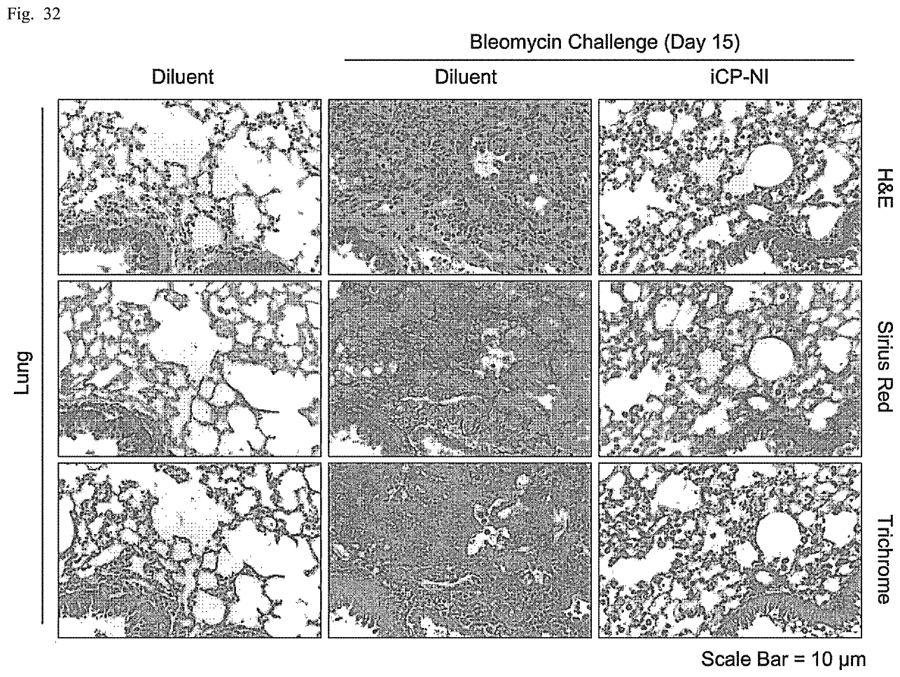

ARDS and moderate to severe cases SARS-CoV-2 infection are associated with interstitial fibrosis. We used a bleomycin (BLM)-induced mouse model of pulmonary fibrosis to test anti-fibrotic effects of iCP-NI. 15 hours after BLM challenge, the lungs of BLM-treated mice showed widespread alveolar injury, as imaged by micro-CT (FIG. 31), characterized by loss of alveolar structure, inflammatory cell infiltration, hyalinization and collagen deposition (FIG. 32). These fibrotic changes were largely blocked by IV iCN-NI treatment.

Together, these results suggest that pulmonary inflammation responds well to intravenously administered iCP-NI initiated prior to the proinflammatory stimulus. Restricted production of a chemokine MCP-1, results in suppressed chemotaxis of immunocytes, especially neutrophils which are the primary culprits in the cytokine storm observed in moderate to severe COVID-19 disease. Thus, iCP-NI might be an effective COVID-19 treatment with its ability to dampen chemokine/cytokine expression, preserve alveolar structure and suppress systemic inflammation leading to microvascular damage and multi-organ failure.

6. Efficacy of iCP-NI on Cytokine/Chemokine Secretion and Lung Inflammation in SARS-CoV-2-Infected Non-Human Primates

After infecting non-human primates (African green monkey: AGM, rhesus macaque: RM) with SARS-CoV-2, iCP-NI was infused to observe improvement in clinical signs (02 saturation, respiratory rates, heart rates, blood glucose levels, body temperature). Cytokines/chemokines plasma levels and BALF were tracked in all animals. All animals were sacrificed, and histopathological analysis was carried out. Unfortunately, monkeys infected with SARS-CoV-2 showed only mild symptoms and BAL fluid and blood plasma levels of pro-inflammatory cytokine/chemokines were low.

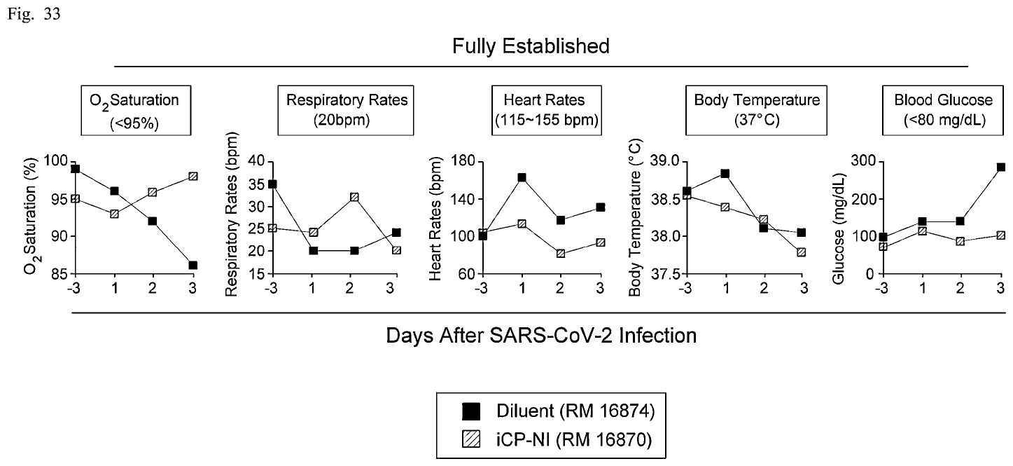

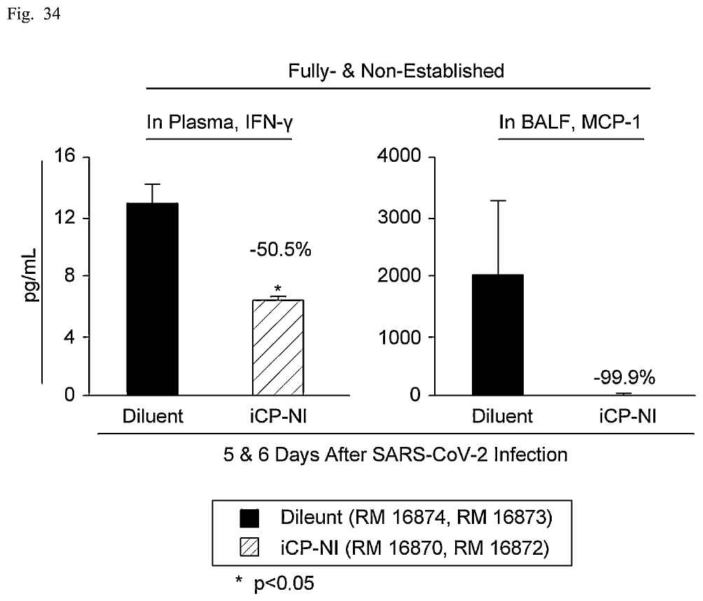

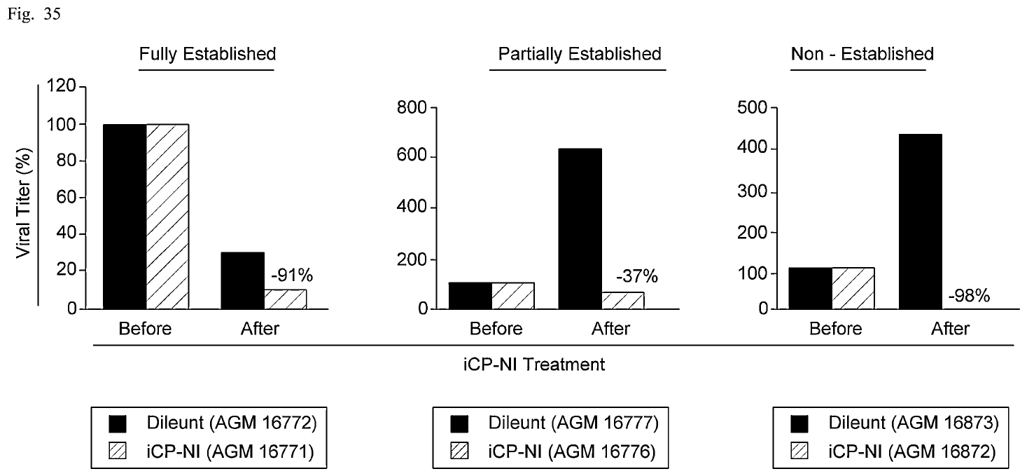

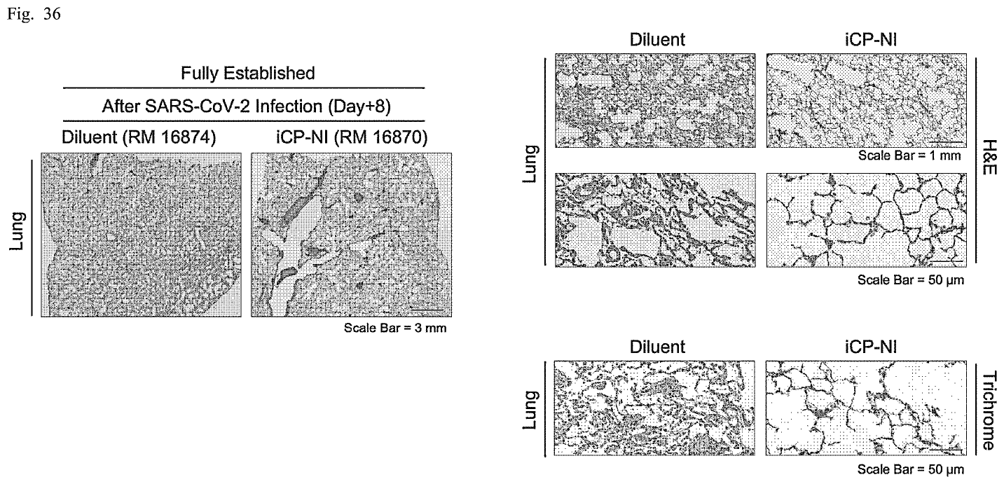

Despite the mild symptoms and low cytokines/chemokines secretion in SARS-CoV-2 infected monkeys, clinical symptoms were improved and secretion of cytokines (IFN-γ)/chemokine (MCP-1) were decreased in iCP-NI-treated animals. On 1 to 5 dpi (day post infection), 02 saturation was dropped to 86%, respiratory rate was increased to 32 times per minute, heart rate rose to 163 bpm, and blood glucose was increased to 286 mg/dL, while iCP-NI-administered monkeys maintained normal condition (oxygen saturation 98%, respiratory rate 20, heart rate 113 times, blood glucose level 103 mg/dL) (FIG. 33). Secretion level of IFN-γ was increased to 12.91 pg/mL in blood plasma, which was subsequently decreased by 50.5% to 6.39 pg/mL following iCP-NI administration. In BALF, the site of direct infection and inflammation, the MCP-1 escalated up to 2010.9 pg/mL 5 days following infection, but was decreased by 99.9% to 24.87 pg/mL in the iCP-NI-treated animals (FIG. 34). Viral titers of SARS-CoV-2 was not correlated with level of clinical symptoms but, its rate of extinction was highly conserved with iCP-NI treatment. Compared with the first day of iCP-NI administration (before treatment), viral titers of diluent animal was only showed 70% of reduction rate, while the iCP-NI treated animal showing 91% of reduction rate in fully established SARS-CoV-2 monkey model. Similarly, diluent animals in partially and non-established group showed increased fold of viral titers (641%, 426%, respectively) while the iCP-NI treated animals are showing 37% and 98% of reduction rate, respectively (FIG. 35). Immune cell infiltration, hyperplasia, hemorrhage and fibroplasia were observed in the lungs of animals without iCP-NI treatment but, these pathological findings were not observed in the iCP-NI treated animals (FIG. 36).

The present disclosure has been described with reference to exemplary embodiments thereof. It will be understood by those skilled in the art to which the present disclosure pertains that the present disclosure may be implemented in modified forms without departing from the spirit and scope of the present disclosure. Therefore, exemplary embodiments disclosed herein should be considered in an illustrative aspect rather than a restrictive aspect. The scope of the present disclosure should be defined by the claims rather than the above-mentioned description, and it shall be interpreted that all differences within the equivalent scope are included in the present disclosure.

### INDUSTRIAL APPLICABILITY

The improved cell-permeable nuclear import inhibitor synthetic peptide according to the present disclosure more efficiently blocks signal transduction mediated by stress-responsive transcription factors (SRTFs) including NF-κB, based on remarkable cell permeability, and thus it may be used as an excellent prophylactic or therapeutic agent for cytokine storm or inflammatory diseases.

### REFERENCE

1. M. E. Starr et al., A new cecal slurry preparation protocol with improved longterm reproducibility for animal models of sepsis. PLoS One 9, e115705 (2014).
2. J. L. Wynn et al., Increased mortality and altered immunity in neonatal sepsis produced by generalized peritonitis. Shock 28, 675-683 (2007).
3. B. Shrum et al., A robust scoring system to evaluate sepsis severity in an animal model. BMC Res Notes 7, 233 (2014).
4. L. Dejager, I. Pinheiro, E. Dejonckheere, C. Libert, Cecal ligation and puncture: the gold standard model for polymicrobial sepsis Trends Microbiol 19, 198-208 (2011).
5. M. G. Toscano, D. Ganea, A. M. Gamero, Cecal ligation puncture procedure. J Vis Exp, (2011).
6. S. Ruiz et al., Sepsis modeling in mice: ligation length is a major severity factor in cecal ligation and puncture. 4, 1-13 (2016).
7. G. Izbicki, M. J. Segel, T. G. Christensen, M. W. Conner, R. Breuer, Time course of bleomycin-induced lung fibrosis. Int J Exp Pathol 83, 111-119 (2002).
8. V. Della Latta, A. Cecchettini, S. Del Ry, M. A. Morales, Bleomycin in the setting of lung fibrosis induction: From biological mechanisms to counteractions. Pharmacol Res 97, 122-130 (2015).

1. An improved cell-permeable nuclear import inhibitor (iCP-NI) synthetic peptide for inhibition of cytokine storm or an inflammatory disease, the iCP-NI synthetic peptide comprising: an NF-κB nuclear localization sequence (NLS) and an advanced macromolecule transduction domain (aMTD), wherein the NF-κB nuclear localization sequence includes an amino acid sequence of SEQ ID NO: 1, and the advanced macromolecule transduction domain includes an amino acid sequence selected from the group consisting of SEQ ID NOs: 2 to 6.

2. The iCP-NI synthetic peptide of claim 1, wherein the NF-κB nuclear localization sequence is a linear NF-κB nuclear localization sequence or circular NLS with two additional cysteine.

3. The iCP-NI synthetic peptide of claim 1, wherein the iCP-NI synthetic peptide inhibits nuclear transport of stress-responsive transcription factor (SRTFs).

4. The iCP-NI synthetic peptide of claim 3, wherein the stress-responsive transcription factor is NF-κB (nuclear factor KB), NFAT (nuclear factor of activated T cells), AP1 (activator protein 1), STAT1 (signal transducer and activator of transcription 1), or Nrf2 (nuclear factor erythroid 2-related factor 2).

5. The iCP-NI synthetic peptide of claim 1, wherein the inflammatory disease includes an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease.

6. The iCP-NI synthetic peptide of claim 5, wherein the autoimmune disease includes rheumatoid arthritis, psoriasis, atopic dermatitis, Crohn's disease, inflammatory bowel disease, Sjorgen's syndrome, optic neuritis, chronic obstructive pulmonary disease, asthma, type I diabetes, neuromyelitis optica, Myasthenia Gavis, uveitis, Guillain-Barre syndrome, psoriatic arthritis, Gaves' disease or allergy.

7. A pharmaceutical composition for preventing or treating cytokine storm or an inflammatory disease, the pharmaceutical composition comprising the iCP-NI synthetic peptide of claim 1.

8. The pharmaceutical composition of claim 7, wherein the cytokine storm or inflammatory disease is induced from inflammatory infections caused by viruses, bacteria, fungi, or parasites.

9. The pharmaceutical composition of claim 8, wherein the viruses include coronavirus, influenza virus, Hantavirus, flavivirus, Epstein-Barr virus, human immunodeficiency virus, Ebola virus, retrovirus, or variola virus.

10. The pharmaceutical composition of claim 8, wherein the bacterial infection includes bacteremia, bacterial sepsis, pneumonia, cellulitis, meningitis, erysipelas, infective endocarditis, necrotizing fasciitis, prostatitis, pseudomembranous colitis, pyelonephritis, or septic arthritis.

11. The pharmaceutical composition of claim 8, wherein the fungi include Aspergillis, Candida albicans, or Cryptococcus neoformans.

12. The pharmaceutical composition of claim 8, wherein the parasites include Plasmodium falciparum.

13. The pharmaceutical composition of claim 7, further comprising an antibiotic, anti-viral agent, anti-HIV agent, anti-parasite agent, anti-protozoal agent, steroidal agent, steroidal or non-steroidal anti-inflammatory agent, antihistamine, immunosuppressant agent, or a combination thereof.

14. The pharmaceutical composition of claim 13, wherein the antibiotic includes cephalosporin series, beta-lactam series, beta-lactam/beta-lactamase inhibitor series, quinolone series, glycopeptide series, carbapenem series, aminoglycoside series, macrolide series, sulfa drug series, aztreonam, clindamycin, tigecycline, colistin sodium methanesulfonate, metronidazole, spiramycin, or a combination thereof.

15. The pharmaceutical composition of claim 7, wherein the inflammatory disease includes an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease.

16. The pharmaceutical composition of claim 15, wherein the autoimmune disease includes rheumatoid arthritis, psoriasis, atopic dermatitis, Crohn's disease, inflammatory bowel disease, Sjorgen's syndrome, optic neuritis, chronic obstructive pulmonary disease, asthma, type I diabetes, neuromyelitis optica, Myasthenia Gavis, uveitis, Guillain-Barre syndrome, psoriatic arthritis, Gaves' disease or allergy.

17. A method of preventing or treating cytokine storm or an inflammatory disease, the method comprising the step of administering, to a subject, the iCP-NI synthetic peptide of claim 1.

18. The method of claim 17, wherein the iCP-NI synthetic peptide is co-administered with an antibiotic, anti-viral agent, anti-HIV agent, anti-parasite agent, anti-protozoal agent, steroidal agent, steroidal or non-steroidal anti-inflammatory agent, antihistamine, immunosuppressant agent, or a combination thereof.

19. The method of claim 18, wherein the antibiotic includes cephalosporin series, beta-lactam series, beta-lactam/beta-lactamase inhibitor series, quinolone series, glycopeptide series, carbapenem series, aminoglycoside series, macrolide series, sulfa drug series, aztreonam, clindamycin, tigecycline, colistin sodium methanesulfonate, metronidazole, spiramycin, or a combination thereof.

20. The method of claim 17, wherein the administration includes intravenous, parenteral, transdermal, subcutaneous, intramuscular, intracranial, intraorbital, intraocular, intraventricular, intracapsular, intrathecal, intracisternal, intraperitoneal, intranasal, intrarectal, intravaginal, spraying, or oral administration.

21. The method of claim 17, wherein the cytokine storm or inflammatory disease is induced from inflammatory infections caused by viruses, bacteria, fungi, or parasites.

22. The method of claim 17, wherein the cytokine storm or inflammatory disease is induced by trauma, injury, burns, toxins, or carcinogens.

23. The method of claim 17, wherein the inflammatory disease includes an autoimmune disease, graft rejection, multiple sclerosis, pancreatitis, acute bronchitis, chronic bronchitis, acute bronchiolitis, chronic bronchiolitis, sepsis, septic shock, acute respiratory distress syndrome, multiple organ failure, or chronic obstructive pulmonary disease.
